SEQUENCE LISTING (62 sequences)

SEQ ID NO: 1 - protein, 5 aa, Artificial Sequence
  Description of Artificial Sequence Synthetic peptide
GVAFG

SEQ ID NO: 2 - protein, 8 aa, Artificial Sequence
  Description of Artificial Sequence Synthetic peptide
QWAVGHXL

SEQ ID NO: 3 - protein, 8 aa, Artificial Sequence
  Description of Artificial Sequence Synthetic peptide
QWAVGHXL

SEQ ID NO: 4 - protein, 8 aa, Artificial Sequence
  Description of Artificial Sequence Synthetic peptide
QWAVGHXL

SEQ ID NO: 5 - protein, 8 aa, Artificial Sequence
  Description of Artificial Sequence Synthetic peptide
QWAVGHXL

SEQ ID NO: 6 - protein, 8 aa, Artificial Sequence
  Description of Artificial Sequence Synthetic peptide
QWAVGHXX

SEQ ID NO: 7 - protein, 8 aa, Artificial Sequence
  Description of Artificial Sequence Synthetic peptide
QWAVGHXL

SEQ ID NO: 8 - protein, 8 aa, Artificial Sequence
  Description of Artificial Sequence Synthetic peptide
QWAVGHXL

SEQ ID NO: 9 - protein, 8 aa, Artificial Sequence
  Description of Artificial Sequence Synthetic peptide
QWAVGHXL

SEQ ID NO: 10 - protein, 8 aa, Artificial Sequence
  Description of Artificial Sequence Synthetic peptide
QWAVGHXX

SEQ ID NO: 11 - protein, 8 aa, Artificial Sequence
  Description of Artificial Sequence Synthetic peptide
QWAVGHXX

SEQ ID NO: 12 - protein, 8 aa, Artificial Sequence
  Description of Artificial Sequence Synthetic peptide
QWAVGHXG

SEQ ID NO: 13 - protein, 8 aa, Artificial Sequence
  Description of Artificial Sequence Synthetic peptide
QWAVGHXG

SEQ ID NO: 14 - protein, 8 aa, Artificial Sequence
  Description of Artificial Sequence Synthetic peptide
QWAVGHXL

SEQ ID NO: 15 - protein, 8 aa, Artificial Sequence
  Description of Artificial Sequence Synthetic peptide
QWAVGHXG

SEQ ID NO: 16 - protein, 8 aa, Artificial Sequence
  Description of Artificial Sequence Synthetic peptide
QWAVGHXX

SEQ ID NO: 17 - protein, 8 aa, Artificial Sequence
  Description of Artificial Sequence Synthetic peptide
QWAVGHXX

SEQ ID NO: 18 - protein, 8 aa, Artificial Sequence
  Description of Artificial Sequence Synthetic peptide
QWAVGHXA

SEQ ID NO: 19 - protein, 8 aa, Artificial Sequence
  Description of Artificial Sequence Synthetic peptide
QWAVGHXX

SEQ ID NO: 20 - protein, 8 aa, Artificial Sequence
  Description of Artificial Sequence Synthetic peptide
QWAVGHXG

SEQ ID NO: 21 - protein, 8 aa, Artificial Sequence
  Description of Artificial Sequence Synthetic peptide
QWAVGHXL

SEQ ID NO: 22 - protein, 8 aa, Artificial Sequence
  Description of Artificial Sequence Synthetic peptide
QWAVGHXL

SEQ ID NO: 23 - protein, 8 aa, Artificial Sequence
  Description of Artificial Sequence Synthetic peptide
QWAVGHXX

SEQ ID NO: 24 - protein, 8 aa, Artificial Sequence
  Description of Artificial Sequence Synthetic peptide
QWAVGHXL

SEQ ID NO: 25 - protein, 8 aa, Artificial Sequence
  Description of Artificial Sequence Synthetic peptide
QWAVGHXL

SEQ ID NO: 26 - protein, 8 aa, Artificial Sequence
  Description of Artificial Sequence Synthetic peptide
QWAVGHXL

SEQ ID NO: 27 - protein, 8 aa, Artificial Sequence
  Description of Artificial Sequence Synthetic peptide
QWAVAHXX

SEQ ID NO: 28 - protein, 8 aa, Artificial Sequence
  Description of Artificial Sequence Synthetic peptide
QWAVAHFX

SEQ ID NO: 29 - protein, 8 aa, Artificial Sequence
  Description of Artificial Sequence Synthetic peptide
QWAVAHFL

SEQ ID NO: 30 - protein, 8 aa, Artificial Sequence
  Description of Artificial Sequence Synthetic peptide
QWAVAHFL

SEQ ID NO: 31 - protein, 8 aa, Artificial Sequence
  Description of Artificial Sequence Synthetic peptide
QWAVAHLL

SEQ ID NO: 32 - protein, 8 aa, Artificial Sequence
  Description of Artificial Sequence Synthetic peptide
QWAVAHIL

SEQ ID NO: 33 - protein, 8 aa, Artificial Sequence
  Description of Artificial Sequence Synthetic peptide
QWAVAHLG

SEQ ID NO: 34 - protein, 8 aa, Artificial Sequence
  Description of Artificial Sequence Synthetic peptide
QWAVAHFX

SEQ ID NO: 35 - protein, 8 aa, Artificial Sequence
  Description of Artificial Sequence Synthetic peptide
QWAVAHFX

SEQ ID NO: 36 - protein, 8 aa, Artificial Sequence
  Description of Artificial Sequence Synthetic peptide
QWAVAHFG

SEQ ID NO: 37 - protein, 8 aa, Artificial Sequence
  Description of Artificial Sequence Synthetic peptide
QWAVAHXG

SEQ ID NO: 38 - protein, 8 aa, Artificial Sequence
  Description of Artificial Sequence Synthetic peptide
QWAVAHXG

SEQ ID NO: 39 - protein, 8 aa, Artificial Sequence
  Description of Artificial Sequence Synthetic peptide
QWAVAHFX

SEQ ID NO: 40 - protein, 8 aa, Artificial Sequence
  Description of Artificial Sequence Synthetic peptide
QWAVAHFL

SEQ ID NO: 41 - protein, 8 aa, Artificial Sequence
  Description of Artificial Sequence Synthetic peptide
QWAVAHFL

SEQ ID NO: 42 - protein, 8 aa, Artificial Sequence
  Description of Artificial Sequence Synthetic peptide
QWAVAHFL

SEQ ID NO: 43 - protein, 8 aa, Artificial Sequence
  Description of Artificial Sequence Synthetic peptide
QWAVAHFL

SEQ ID NO: 44 - protein, 8 aa, Artificial Sequence
  Description of Artificial Sequence Synthetic peptide
QWAVAHFL

SEQ ID NO: 45 - protein, 8 aa, Artificial Sequence
  Description of Artificial Sequence Synthetic peptide
QWAVAHFL

SEQ ID NO: 46 - protein, 8 aa, Artificial Sequence
  Description of Artificial Sequence Synthetic peptide
QWAVAHIG

SEQ ID NO: 47 - protein, 8 aa, Artificial Sequence
  Description of Artificial Sequence Synthetic peptide
QWAVGHXX

SEQ ID NO: 48 - protein, 8 aa, Artificial Sequence
  Description of Artificial Sequence Synthetic peptide
QWAVGHXX

SEQ ID NO: 49 - protein, 8 aa, Artificial Sequence
  Description of Artificial Sequence Synthetic peptide
QWAVGHXA

SEQ ID NO: 50 - protein, 8 aa, Artificial Sequence
  Description of Artificial Sequence Synthetic peptide
QWAVGHXA

SEQ ID NO: 51 - protein, 7 aa, Artificial Sequence
  Description of Artificial Sequence Synthetic peptide
QWAVHXL

SEQ ID NO: 52 - protein, 8 aa, Artificial Sequence
  Description of Artificial Sequence Synthetic peptide
QWAVGHXL

SEQ ID NO: 53 - protein, 8 aa, Artificial Sequence
  Description of Artificial Sequence Synthetic peptide
QWAVGHXL

SEQ ID NO: 54 - protein, 8 aa, Artificial Sequence
  Description of Artificial Sequence Synthetic peptide
QWAVGHXL

SEQ ID NO: 55 - protein, 9 aa, Artificial Sequence
  Description of Artificial Sequence Synthetic peptide
QWAVGHXXL

SEQ ID NO: 56 - protein, 9 aa, Artificial Sequence
  Description of Artificial Sequence Synthetic peptide
QWAVGHXXX

SEQ ID NO: 57 - protein, 6 aa, Artificial Sequence
  Description of Artificial Sequence Synthetic peptide
KFXWKT

SEQ ID NO: 58 - protein, 6 aa, Artificial Sequence
  Description of Artificial Sequence Synthetic peptide
XMFYWK

SEQ ID NO: 59 - protein, 8 aa, Artificial Sequence
  Description of Artificial Sequence Synthetic peptide
CLITRCRY

SEQ ID NO: 60 - protein, 8 aa, Artificial Sequence
  Description of Artificial Sequence Synthetic peptide
CLIVRCRY

SEQ ID NO: 61 - protein, 4 aa, Artificial Sequence
  Description of Artificial Sequence Synthetic peptide
VAFG

SEQ ID NO: 62 - protein, 4 aa, Artificial Sequence
  Description of Artificial Sequence Synthetic peptide
VAHG

(19) United States
(12) Patent Application Publication
(10) Pub. No.: US 2009/0022664 A1
(43) Pub. Date: Jan. 22, 2009

(54) Radiolabelling via fluorination of aziridines

(76) Inventors: Ananth Srinivasan, Berlin (DE); Thomas Brumby, Berlin (DE); Timo Stellfeld, Berlin (DE); Keith Graham, Berlin (DE); Ulrike Voigtmann, Berlin (DE); Jessica Becaud, Olten (CH); Linjing Mu, Allschwill (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/851,786

(22) Filed: Sep. 7, 2007

Related U.S. Application Data
(60) Provisional application No. 60/880,010, filed on Jan. 12, 2007.
(60) Provisional application No. 60/914,886, filed on Apr. 30, 2007.

(30) Foreign Application Priority Data
Jan. 9, 2007 (EP) 07090001.4
Apr. 23, 2007 (EP) 07090079.0

(51) Int. Cl. A61K 51/08; C07D 203/08; C07D 403/12; C07K 7/06; A61K 51/04; C07H 19/073; C07D 487/04

(52) U.S. Cl. 424/1.69; 548/966; 548/518; 530/330; 536/28.2; 548/453; 424/1.73; 424/1.89

(57) ABSTRACT

The present invention relates to novel compounds suitable for or already radiolabelled with ¹⁸F, methods of making such compounds and use of such compounds for diagnostic imaging.

This application claims the benefit of the filing date of U.S. Provisional Application Nos. 60/880,010 filed Jan. 12, 2007 and 60/914,886, filed Apr. 30, 2007, which are incorporated by reference herein.

### FIELD OF INVENTION

This invention relates to novel compounds suitable for labelling or already labelled with an appropriate fluorine isotope, preferably ¹⁸F, methods of preparing such compounds, compositions comprising such compounds, kits comprising such compounds or compositions and uses of such compounds, compositions or kits for diagnostic imaging, preferably for positron emission tomography (PET).

### BACKGROUND ART

Molecular imaging has the potential to detect disease progression or therapeutic effectiveness earlier than most conventional methods in the fields of oncology, neurology and cardiology. Of the several promising molecular imaging technologies having been developed as optical imaging and MRI, Positron Emission Tomography (PET) is of particular interest for drug development because of its high sensitivity and ability to provide quantitative and kinetic data.

Over the last few years, in vivo scanning using PET has increased. PET is both a medical and research tool. It is used heavily in clinical oncology for medical imaging of tumors and the search for metastases, and for clinical diagnosis of certain diffuse brain diseases such as those causing various types of dementias. Radiotracers consisting of a radionuclide stably bound to a biomolecule is used for in vivo imaging of disorders.

In designing an effective radiopharmaceutical tracer for use as a diagnostic agent, it is imperative that the drugs have appropriate in vivo targeting and pharmacokinetic properties. Fritzberg et al., J Nucl. Med., 1992, 33:394, further state that radionuclide chemistry and associated linkages underscore the need to optimize attachment and labelling chemical modifications of the biomolecule carrier. Hence the type of radionuclide, the type of biomolecule and the method used for linking them to one another may have a crucial effect onto the radiotracer properties.

The radionuclides used in PET scanning are typically isotopes with short half lives such as ¹¹C (˜20 min), ¹³N (˜10 min), ¹⁵O (˜2 min), ⁶⁸Ga (˜68 min) or ¹⁸F (˜110 min). Due to their short half lives, the radionuclides must be produced in a cyclotron which is not too far away in delivery-time from the PET scanner. These radionuclides are incorporated into biologically active compounds or biomolecules that have the function to vehicle the radionuclide into the body to the targeted site, e.g., to the tumor.

Positron emitting isotopes include carbon, nitrogen, and oxygen. These isotopes can replace their non-radioactive counterparts in target compounds to produce tracers that function biologically and are chemically identical to the original molecules for PET imaging. On the other hand, ¹⁸F is the most convenient labelling isotope due to its relatively long half life (109.6 min) which permits the preparation of diagnostic tracers and subsequent study of biochemical processes. In addition, its low β+ energy (635 keV) is also advantageous.

PET tracers are or often include a molecule of biological interest. Biomolecules developed for use in PET have been numerously intended for specific targeting in the patient as, e.g., FDG, FLT, L-DOPA, methionine and deoxythymidine. Due to their specific use, such biomolecules are often designated as “targeting agents”.

Peptides are biomolecules that play a crucial role in many physiological processes including actions as neurotransmitters, hormones and antibiotics. Research has shown their importance in such fields as neuroscience, immunology, pharmacology, and cell biology. Some peptides can act as chemical messenger. They bind to receptor on the target cell surface and the biological effect of the ligand is transmitted to the target tissue. Hence the specific receptor binding property of the ligand can be exploited by labelling the ligand with a radionuclide. Theoretically, the high affinity of the ligand for the receptor facilitates retention of the radio labelled ligand in receptor expressing tissues. However, it is still under investigation which peptides can be efficiently labelled and under which conditions the labelling shall occur. It is well known that the receptor specificity of a ligand peptide may be altered during chemical reaction. Therefore an optimal peptidic construct has to be determined.

Tumors overexpress various receptor types to which peptides bound specifically. Boerman et al., Seminar in Nuclear Medicine, July, 2000, 30, (3); 195-208, provide a non exhaustive list of peptides binding to receptors involved in tumors, i.e., somatostatin, vasoactive intestinal peptide (VIP), bombesin binding to gastrin-releasing peptide (GRP) receptor, gastrin, cholecystokinin (CCK), and calcitonin.

The linkage of the radionuclide to the biomolecule is done by various methods resulting to the presence or not of a linker between the radionuclide and the biomolecule. Hence, various linkers are known. C. J. Smith et al., “Radiochemical investigations of ¹⁷⁷Lu-DOTA-8-Aoc-BBN[7-14]NH2: an in vitro/in vivo assessment of the targeting ability of this new radiopharmaceutical for PC-3 human prostate cancer cells.” Nucl. Med. Bio., 2003, 30(2):101-9, disclose radiolabelled bombesin wherein the linker is DOTA-X where X is a carbon tether. However, the radiolabel ¹⁷⁷Lu (half life 6.5 days) does not match the biological half-life of the native bombesin what makes the ¹⁷⁷Lu-DOTA-X-bombesin a non-appropriate radiotracer for imaging tumors.

E. Garcia Garayoa et al., “Chemical and biological characterization of new Re(CO)3/[^{99m}Tc](CO)3 bombesin analogues.” Nucl. Med. Biol., 2007:17-28, disclose a spacer between the radionuclide [^{99m}Tc] and the bombesin wherein the spacer is -β-Ala-β-Ala- and 3,6-dioxa-8-aminooctanoic acid. E. Garcia Garayoa et al. conclude that the different spacer did not have a significant effect on stability or on receptor affinity.

Listed above linkers have been specifically designed for a specific type of radionuclide and determine the type and chemical conditions of the radiobinding method.

More recently, peptides have been conjugated to macrocyclic chelators for labelling of ⁶⁴Cu, ⁸⁶Y, and ⁶⁸Ga for PET application. However, such radionuclides interact with the in vivo catabolism resulting in unwanted physiologic effects and chelate attachment. Various methods of radiofluorination have been published using different precursor or starting materials for obtaining ¹⁸F-labelled peptides. Due to the smaller size of peptides, both higher target-to-background ratios and rapid blood clearance can often be achieved with radiolabelled peptides. Hence, short-lived positron emission tomography (PET) isotopes are potential candidates for labelling peptides. Among a number of positron-emitting nuclides, fluorine-18 appears to be the best candidate for labelling bioactive peptides by virtue of its favourable physical and nuclear characteristics. The major disadvantage of labelling peptides with ¹⁸F is the laborious and time-consuming preparation of the ¹⁸F labelling agents. Due to the complex nature of peptides and several functional groups associated with the primary structure, ¹⁸F-labelled peptides are not prepared by direct fluorination. Hence, difficulties associated with the preparation of ¹⁸F-labelled peptides were alleviated with the employment of prosthetic groups as shown below. Several such prosthetic groups have been proposed in the literature, including N-succinimidyl-4-[¹⁸F] fluorobenzoate, m-maleimido-N-(p-[¹⁸F]fluorobenzyl)-benzamide, N-(p-[¹⁸F]fluorophenyl) maleimide, and 4-[¹⁸F]fluorophenacylbromide. Almost all of the methodologies currently used today for the labelling of peptides and proteins with ¹⁸F utilize active esters of the fluorine labelled synthon.

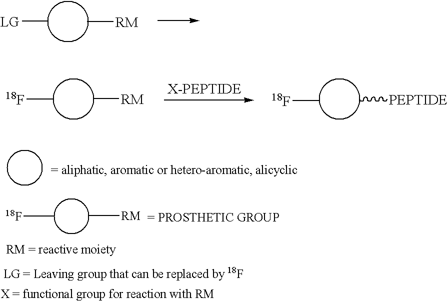

Okarvi et al., “Recent progress in fluorine-18 labelled peptide radiopharmaceuticals.” Eur. J. Nucl. Med., July 2001, 28(7):929-38, present a review of the recent developments in ¹⁸F-labelled biologically active peptides used in PET.

Zhang Xianzhong et al., “¹⁸F-labelled bombesin analogs for targeting GRP receptor-expressing prostate cancer.” J. Nucl. Med., 2006, 47(3):492-501, relate to the 2-step method detailed above. [Lys3]Bombesin ([Lys3]BBN) and aminocaproic acid-bombesin(7-14) (Aca-BBN(7-14)) were labelled with ¹⁸F by coupling the Lys3 amino group and Aca amino group, respectively, with N-succinimidyl-4-¹⁸F-fluorobenzoate (¹⁸F-SFB) under slightly basic condition (pH 8.5). Unfortunately, the obtained ¹⁸F-FB-[Lys3]BBN is relatively metabolically unstable having for result to reduce the extent of use of the ¹⁸F-FB-[Lys3]BBN for reliable imaging of tumors.

Poethko Thorsten et al., “Two-step methodology for high-yield routine radiohalogenation of peptides: ¹⁸F-labelled RGD and octreotide analogs.” J. Nucl Med., May 2004, 45(5):892-902, relate to a 2-step method for labelling RGD and octreotide analogs. The method discloses the steps of radiosynthesis of the ¹⁸F-labelled aldehyde or ketone and the chemoselective ligation of the ¹⁸F-labelled aldehyde or ketone to the aminooxy functionalized peptide.

Poethko Thorsten et al., “First ¹⁸F-labelled tracer suitable for routine clinical imaging of somatostatin receptor-expressing tumors using positron emission tomography.” Clin. Cancer Res., June 2004, 1, 10(11):3593-606, apply the 2-step method for the synthesis of ¹⁸F-labelled carbohydrated Tyr(3)-octreotate (TOCA) analogs with optimized pharmacokinetics suitable for clinical routine somatostatin-receptor (sst) imaging.

WO 2003/080544 A1 and WO 2004/080492 A1 relate to radiofluorination methods of bioactive peptides for diagnostics imaging using the 2-step method shown above.

¹⁸F-labelled compounds are gaining importance due to their availability as well as due to the development of methods for labelling biomolecules. It has been shown that some compounds labelled with ¹⁸F, produce images of high quality. Additionally, the longer lifetime of ¹⁸F would permit longer imaging times and allows preparation of radiotracer batches for multiple patients and delivery of the tracer to other facilities, making the technique more widely available to clinical investigators. Additionally, it has been observed the development of PET cameras and availability of the instrumentation in many PET centers is increasing. Hence, it is increasingly important to develop new tracers labelled with ¹⁸F.

Several approaches for incorporating ¹⁸F into more complex biomolecules as, e.g., peptides are described in the following references: European J. Nucl. Med. Mol. Imaging, 2001, 28:929-938; European J. Nucl. Med. Mol. Imaging, 2004, 31:1182-1206; Bioconjugate Chem., 1991, 2:44-49; Bioconjugate Chem., 2003, 14:1253-1259.

These methods are indirect. They demand at least a two step procedure for tracer synthesis. Therefore they are time consuming thereby reducing PET image resolution as a result of nuclear decay.

The most crucial aspect in the successful treatment of any cancer is early detection. Likewise, it is crucial to properly diagnose the tumors and metastases.

Routine application of ¹⁸F-labelled peptides for quantitative in vivo receptor imaging of receptor-expressing tissues and quantification of receptor status using PET is limited by the lack of appropriate radiofluorination methods for routine large-scale synthesis of ¹⁸F-labelled peptides. There is a clear need for radiofluorination method that can be conducted rapidly without loss of receptor affinity by the peptide and leading to a positive imaging (with reduced background), wherein the radiotracer is stable and shows enhanced clearance properties.

Very few publications are known which describe the opening of aziridines by ¹⁸F:

L. Tron et al. present the reaction of an acyl-activated aziridine moiety with ¹⁸F⁻ at 120° C. in the synthesis of [¹⁸F]FNECA as an adenosine receptor labelling agent. The desired product was obtained with a yield of 1%. The precursor carrying the aziridine remained mainly unreacted. (Journal of Labelled Compounds and Radiopharmaceuticals, 2000, 43:807-815.) We surprisingly found that by a different activation of the aziridine, complete conversion at much lower temperatures towards the desired ring-opened product can be observed.

W. Feindel et al., synthesized [¹⁸F]BFNU and [¹⁸F]CFNU, analogues of the chemotherapeutic drug BCNU, by nucleophilic attack of ¹⁸F-TBAF at 100 or 145° C. on the aziridine ring of 1,3-substituted ureas in rather low yields. (Canadian Journal Chemistry, 1984, 62:2107-2112).

The mentioned aziridine precursors cannot be coupled to chemical functionalities like amines, thiols, hydroxyls, carboxylic acid functions or other chemical groups of complex targeting agents without further transformations as it is achieved herein.

Furthermore, the high temperatures used are not applicable to sensitive bioactive molecules as peptides used as targeting agents herein.

Even publications about cold fluorinations are at a manageable quantity and rather performed with
BF₃.OEt₂: Synlett 2004, 12:2218-2220.; Recl Trav. Chim. Pays-Bas 1992, 111(2):59-68.;
HF* Pyridine (Olah's Reagent): Journal of Chemical Research, Synopses 1983, 10:246-7.; Journal of the Chemical Society, Perkin Transactions 1: Organic and Bio-Organic Chemistry, 1983, 9:2045-51.; Journal of Organic Chemistry, 1981, 46(24):4938-48.; Journal of Fluorine Chemistry, 1980, 16(3):277-83.; Journal of Fluorine Chemistry, 1980, 16(2):183-7.; Journal of Organic Chemistry, 1980, 45(26):5328-33.; Tetrahedron Letters, 1980, 21(3):289-92.; Journal of Fluorine Chemistry, 1990, 49(2):231-46.; Tetrahedron, 1987, 43(11):2485-92.; Tetrahedron Letters, 1978, 35:3247-50.; Journal of Fluorine Chemistry, 1981, 18:93-96.; Journal of Fluorine Chemistry, 1980, 16:277-84.; Journal of Medicinal Chemistry, 1990, 33(9):2603-2610.; Journal of Fluorine Chemistry, 1980, 16:538-539.;
Pyridiniumpolyhydrogenfluoride: Journal of Fluorine Chemistry, 1983, 23:481;
DAST: Tetrahedron, 1999, 55(48):13819-13830; or
LiBF₄: Journal of Organic Chemistry, 1989, 54(22):5324-30;
   than with nucleophilic fluorination reagents preferred in ¹⁸F fluorinations such as
TBAF: Carbohydrate Research, 2003, 338(24):2825-34; Journal of Organic Chemistry, 1989, 54(22):5324-5330; Carbohydrate Research, 1980, 83:142-145; Tetrahedron Asymmetry, 2004, 15(20):3307-3322;
KHF₂: Carbohydrate Research, 1992, 230:89-106; or
KF: Journal of Organic Chemistry, 2004, 69(2):335-338.

Preparation of ¹⁸F-labelled 2-fluoroethylamines, -amides and -sulfonamides is normally performed by at least two step procedures applying ¹⁸F-2-fluorethylamine or 2-bromo-fluorethane. Opening of appropriate aziridines may deliver such structural motifs by single step synthesis.

Peptides containing aziridines are described in several publications but the purpose of their synthesis, their substitution pattern and their applications are different from the use as precursor for radioactive labelling claimed herein.

A method for site- and stereoselective peptide modification using aziridine-2-carboxylic acid-containing peptides for site-selective conjugation with various thiol nucleophiles is described.

Journal of the American Chemical Society, 2005, 127(20):7359-7369. Journal of the American Chemical Society, 2004, 126(40):12712-12713.

A ligand with an aziridine-containing side chain designed to mimic arginine and to bind covalently in the arginine-specific P2 pocket of the class I major histocompatibility complex (MHC) glycoprotein HLA-B27 has been synthesized which alkylates specifically cysteine 67. Proceedings of the National Academy of Sciences of the United States of America, 1996, 93(20):10945-10948.

An aziridine containing lysine derivative as a potential LSD1 inhibitor based on structural considerations and in analogy to known strategies for blocking amine oxidases has been prepared. Journal of the American Chemical Society, 2006, 128(14):4536-4537.

Small molecules with aziridines modified peptides have been claimed as antidepressant compounds to treat patients suffering from depression. WO 99/22758 A.

Further, aziridine compounds are disclosed by R. Rocchiccioli et al., “Alcaloides Peptidiques—I. Approche de la synthese des alcaloides peptidiques. 2. Préparation d'ansapeptides à 15, 17 et 18 chainons”, Tetrahedron, 1978, 34:2917-26, to be intermediates in the synthesis of the title compounds.

I. Funaki et al., “Synthesis of 3-aminopyrrolidin-2-ones by an intramolecular reaction of aziridinecarboxamides”, Tetrahedron, 1996, 52:9909-24, disclose N-substituted aziridine carboxamides to yield 4,5-disubstituted-3-amino-γ-lactams.

T. Wakamiya et al., “Synthesis of threo-3-methylsysteine from threonine”, Bull. Chem. Soc. Jpn., 1982, 55(12):3878-81, disclose the reaction of 3-methyl-2-aziridine carboxylic acid derivatives with thiobenzoic acid to yield 3-methylcysteine.

K. Nakayima et al., “Studies on 2-aziridinecarboxylic acid. VII. Formation of dehydroamino acid peptides via isomerization of peptides containing 2-aziridinecarboxylic acid by tertiary amines”, Bull. Chem. Soc. Jpn., 1982, 55(10):323-36, have disclosed dehydrohydantoin derivatives being prepared by treatment of benzyloxycarbonyl-2-aziridinecarboxylic acid derivatives with tertiary amines.

K. Okawa et al., “Studies of hydroxy amino acids. V. Synthesis and N-acylation of 3-methyl-L-azylylglycine benzyl ester”, Chem. Letters, 1975:591-94, disclose aziridine derivatives as intermediates in β-elimination reaction on hydroxy amino acid derivatives.

K. Nakajima et al., “The reaction of peptides containing β-hydroxy-α-amino acid with Mitsunobu reagents”, Peptide Chemistry, 1983, 20:19-24, disclose 2-aziridine carboxylic acid derivatives.

D. Tanner et al., “Nucleophilic ring opening of C₂-symmetric aziridines. Synthetic equivalents for the β-cation of aspartic acid”, Tetrahedron Letters, 1990, 31(13):1903-6; disclose 2,3-aziridine-dicarboxylic esters undergoing nucleophilic attack to yield products formally derived from the β-cation of aspartic acid.

WO 2001/32622 A1 discloses positive modulators of nicotinic receptor agonists comprising (S)-(+)-2-benzyl-1-(p-tolylsulfonyl)aziridine to be fluorinated with HF.

Sz. Lehel et al., “Synthesis of 5′-N-(2-[¹⁸F]Fluorethyl)-carboxamidoadenosine: A promising tracer for investigation of adenosine receptor system by PET technique”, J. Labelled Cpd. and Radiopharm., 2000, 43:807-815, disclose an aziridine precursor to obtain the title compound.

The preparation of reactive peptide ligands containing aziridines used to change the kinetics of binding by reacting with the protein when bound thereby forming covalent peptide ligand-protein complexes has been claimed. WO 98/14208 A.

Therefore it is an object of the present invention, to develop a practical and mild technique for fluoro radiolabelling, in particular ¹⁸F labelling, of complex biomolecules like peptides in only one rather than two or more chemical steps in order to save time, costs and additional purification steps of radioactive compounds and to provide radiofluorination methods for obtaining radiotracer based on receptor specific peptides for the detection of tumors.

### SUMMARY OF THE INVENTION

In a first aspect, the present invention provides novel compounds comprising an aziridine ring being appropriately activated for one preferably step radio-labelling purposes, wherein a targeting agent radical, either directly or via an appropriate linker, is attached to the aziridine ring or to a five-membered carboxyclic or heterocyclic ring which is fused to the aziridine ring. These compounds are precursors for single step radiolabeling, i.e., radiohalogenation, more preferably radiofluorination.

In a second aspect, the present invention relates to compounds obtainable by a ring opening fluorination reaction of the aziridine ring, especially by a fluorine isotope, and to a pharmaceutically acceptable salt of an inorganic or organic acid thereof, a hydrate, complex, ester, amide, solvate and prodrug thereof.

In a third aspect, the present invention is directed to fluorinated, compounds and to pharmaceutically acceptable salts of inorganic or organic acids thereof, hydrates, complexes, esters, amides, solvates and prodrugs thereof.

In a fourth aspect, the present invention relates to a method of preparing such compounds by reacting compounds according to the first aspect of the present invention with an appropriate fluorination, agent under appropriate reaction conditions. Such method comprises the step of reacting a compound having any one of general chemical Formulae I, II and III with fluorinating agent.

In a fifth aspect, the present invention relates to a composition comprising a compound or a pharmaceutically acceptable salt of an inorganic or organic acid thereof, a hydrate, complex, ester, amide, solvate or prodrug thereof according to the first aspect of the present invention or a fluorinated compound or a pharmaceutically acceptable salt of an inorganic or organic acid thereof, a hydrate, complex, ester, amide, solvate or prodrug thereof, including a compound being prepared with the method according to the fourth aspect of the present invention and additionally a pharmaceutically acceptable carrier, diluent, excipient or adjuvant.

In a sixth aspect, the present invention relates to a kit comprising a compound or a pharmaceutically acceptable salt of an inorganic or organic acid thereof, a hydrate, complex, ester, amide, solvate or prodrug thereof according to the first aspect of the present invention (precursor) along with an acceptable carrier, diluent, excipient or adjuvant supplied as a mixture with the precursor or for the manufacture of fluorinated compounds according to the third aspect. In a further aspect, the present invention relates to a kit comprising a fluorinated compound or a pharmaceutically acceptable salt of an inorganic or organic acid thereof, a hydrate, complex, ester, amide, solvate or prodrug thereof according to the third aspect of the present invention or a composition according to the fifth aspect of the present invention, e.g., in powder form, and a container containing an appropriate solvent for preparing a physiologically acceptable solution of said fluorinated compound or salt, hydrate, complex, ester, amide, solvate or prodrug thereof or of said composition for administration thereof to an animal, including a human.

In a seventh aspect, the present invention is directed to the use of any fluorinated compound or salt, hydrate, complex, ester, amide, solvate or prodrug thereof, as defined hereinabove, or of a respective composition or kit, for diagnostic imaging, in particular positron emission tomography. Further, the present invention is directed to a fluorinated compound, more preferably labelled with ¹⁸F isotope, for use as medicament, more preferably for use as diagnostic imaging agent and more preferably for use as imaging agent for positron emission tomography. In another variation of this aspect, the present invention also relates to fluorinated compounds, which are more preferably labelled with ¹⁹F isotope and which have general chemical Formula II, for use in biological assays and chromatographic identification.

In an eighth aspect, the present invention relates to a method of imaging diseases, comprising introducing into a patient a detectable quantity of a labelled compound having any one of general chemical Formulae I-F-A, I-F-B, II-F-A, II-F-B, III-F-A and III-F-B or B and B-A, respectively.

### DETAILED DESCRIPTION OF INVENTION

As used hereinafter in the description of the invention and in the claims, the term “alkyl”, by itself or as part of another group, refers to a straight chain or branched chain alkyl group with 1 to 20 carbon atoms such as, for example methyl, ethyl, propyl, isopropyl, butyl, isobutyl, tert-butyl, pentyl, isopentyl, neopentyl, heptyl, hexyl, decyl. Alkyl groups can also be substituted, such as by halogen atoms, hydroxyl groups, C₁-C₄ alkoxy groups or C₆-C₁₂ aryl groups (which, intern, can also be substituted, such as by 1 to 3 halogen atoms). More preferably alkyl is C₁-C₁₀ alkyl, C₁-C₆ alkyl or C₁-C₄ alkyl.

As used hereinafter in the description of the invention and in the claims, the term “cycloalkyl” by itself or as part of another group, refers to mono- or bicyclic chain of alkyl group with 3 to 20 carbon atoms such as, for example cyclopropyl, cyclobutyl, cyclopentyl, cyclohexyl or cycloheptyl. More preferably cycloalkyl is C₃-C₁₀ cycloalkyl or C₅-C₈ cycloalkyl, most preferably C₆ cycloalkyl.

As used hereinafter in the description of the invention and in the claims, the term “heterocycloalkyl”, by itself or as part of another group, refers to groups having 3 to 20 mono- or bi-ring atoms of a cycloalkyl; and containing carbon atoms and 1, 2, 3 or 4 oxygen, nitrogen or sulfur heteroatoms. More preferably heterocycloalkyl is C₃-C₁₀ heterocycloalkyl, C₅-C₈ heterocycloalkyl or C₅-C₁₄ heterocycloalkyl, most preferably C₆ heterocycloalkyl.

As used hereinafter in the description of the invention and in the claims, the term “aralkyl” refers to aryl-substituted alkyl radicals such as benzyl, diphenylmethyl, triphenylmethyl, phenylethyl, phenylbutyl and diphenylethyl.

As used hereinafter in the description of the invention and in the claims, the terms “aryloxy” refers to aryl groups having an oxygen through which the radical is attached to a nucleus, examples of which are phenoxy.

As used hereinafter in the description of the invention and in the claims, the terms “alkenyl” and “alkynyl” are similarly defined as for alkyl, but contain at least one carbon-carbon double or triple bond, respectively. More preferably C₂-C₆ alkenyl and C₂-C₆ alkynyl.

As used hereinafter in the description of the invention and in the claims, the term “lower unbranched or branched alkyl” shall have the following meaning: a substituted or unsubstituted, straight or branched chain monovalent or divalent radical consisting substantially of carbon and hydrogen, containing no unsaturation and having from one to eight carbon atoms, e.g., but not limited to methyl, ethyl, n-propyl, n-pentyl, 1,1-dimethylethyl (t-butyl), n-heptyl and the like.

As used hereinafter in the description of the invention and in the claims, the terms “aralkenyl” refers to aromatic structure (aryl) coupled to alkenyl as defined above.

As used hereinafter in the description of the invention and in the claims, the terms “alkoxy (or alkyloxy), aryloxy, and aralkenyloxy” refer to alkyl, aryl, and aralkenyl groups respectively linked by an oxygen atom, with the alkyl, aryl, and aralkenyl portion being as defined above.

As used hereinafter in the description of the invention and in the claims, the terms “salts of inorganic or organic acids”, “inorganic acid” and “organic acid” refer to mineral acids, including, but not being limited to: acids such as carbonic, nitric, phosphoric, hydrochloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic, trifluormethansulfonic and sulfanilic acid, respectively.

As used hereinafter in the description of the invention and in the claims, the term “aryl” by itself or as part of another group refers to monocyclic or bicyclic aromatic groups containing from 6 to 12 carbon atoms in the ring portion, preferably 6-10 carbons in the ring portion, such as phenyl, naphthyl or tetrahydronaphthyl.

As used hereinafter in the description of the invention and in the claims, the term “heteroaryl” by itself or as part of another group, refers to groups having 5 to 14 ring atoms; 6, 10 or 14 π (pi) electrons shared in a cyclic array; and containing carbon atoms and 1, 2, 3 or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl and phenoxazinyl groups).

Whenever the term substituted is used, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a pharmaceutical composition. The substituent groups may be selected from halogen atoms, hydroxyl groups, C₁-C₄ alkoxy groups or C₆-C₁₂ aryl groups (which, intern, can also be substituted, such as by 1 to 3 halogen atoms).

As used hereinafter in the description of the invention and in the claims, the term “fluorine isotope” (F) refers to all isotopes of the fluorine atomic element. Fluorine isotope (F) is selected from radioactive or non-radioactive isotope. The radioactive fluorine isotope is selected from ¹⁸F. The non-radioactive “cold” fluorine isotope is selected from ¹⁹F.

As used hereinafter in the description of the invention and in the claims, the term “prodrug” means any covalently bonded compound, which releases the active parent pharmaceutical according to formula II.

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmaco-logical Basis of Therapeutics, 8 ed, McGraw-HiM, Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxy group, such as the hydroxy group on the asymmetric carbon atom, or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference. Prodrugs are characterized by excellent aqueous solubility, increased bioavailability and are readily metabolized into the active inhibitors in vivo.

As used hereinafter in the description of the invention and in the claims, the terms “amino acid sequence” and “peptide” are defined herein as a polyamide obtainable by (poly)condensation of at least two amino acids.

As used hereinafter in the description of the invention and in the claims, the term “amino acid” means any molecule comprising at least one amino group and at least one carboxyl group, but which has no peptide bond within the molecule. In other words, an amino acid is a molecule that has a carboxylic acid functionality and an amine nitrogen having at least one free hydrogen, preferably in alpha position thereto, but no amide bond in the molecule structure. Thus, a dipeptide having a free amino group at the N-terminus and a free carboxyl group at the C-terminus is not to be considered as a single “amino acid” in the above definition. The amide bond between two adjacent amino acid residues which is obtained from such a condensation is defined as “peptide bond”. Optionally, the nitrogen atoms of the polyamide backbone (indicated as NH above) may be independently alkylated, e.g., with C₁-C₆-alkyl, preferably CH₃.

An amide bond as used herein means any covalent bond having the structure

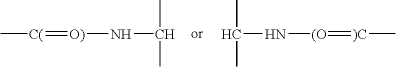

wherein the carbonyl group is provided by one molecule and the NH-group is provided by the other molecule to be joined. The amide bonds between two adjacent amino acid residues which are obtained from such a polycondensation are defined as “peptide bonds”. Optionally, the nitrogen atoms of the polyamide backbone (indicated as NH above) may be independently alkylated, e.g., with —C₁-C₆-alkyl, preferably —CH₃.

As used hereinafter in the description of the invention and in the claims, an amino acid residue is derived from the corresponding amino acid by forming a peptide bond with another amino acid.

As used hereinafter in the description of the invention and in the claims, an amino acid sequence may comprise naturally occurring and/or synthetic/artificial amino acid residues, proteinogenic and/or non-proteinogenic amino acid residues. The non-proteinogenic amino acid residues may be further classified as (a) homo analogues of proteinogenic amino acids, (b) β-homo analogues of proteinogenic amino acid residues and (c) further non-proteinogenic amino acid residues.

Accordingly, the amino acid residues may be derived from the corresponding amino acids, e.g., from
proteinogenic amino acids, namely Ala, Arg, Asn, Asp, Cys, Gln, Glu, Gly, His, Ile, Leu, Lys, Met, Phe, Pro, Ser, Thr, Trp, Tyr and Val; or
non-proteinogenic amino acids, such as
   homo analogues of proteinogenic amino acids wherein the sidechain has been extended by a methylene group, e.g., homoalanine (Hal), homoarginine (Har), homocysteine (Hcy), homoglutamine (Hgl), homohistidine (Hhi), homoisoleucine (Hil), homoleucine (Hle), homolysine (Hly), homomethionine (Hme), homophenylalanine (Hph), homoproline (Hpr), homoserine (Hse), homothreonine (Hth), homotryptophane (Htr), homotyrosine (Hty) and homovaline (Hva);
   β-homo analogues of proteinogenic amino acids wherein a methylene group has been inserted between the α-carbon and the carboxyl group yielding β-amino acids, e.g., β-homoalanine (βHal), β-homoarginine (βHar), β-homoasparagine (βHas), β-homocysteine (βHcy), β-homoglutamine (βHgl), β-homohistidine (βHhi), β-homoisoleucine (βHil), β-homoleucine (βHle), β-homolysine (βHly), β-homomethionine (βHme), β-homophenylalanine (βHph), β-homoproline (βHpr), β-homoserine (βHse), β-homothreonine (βHth), β-homotryptophane (βHtr), β-homotyrosine (βHty) and β-homovaline (βHva);
   further non-proteinogenic amino acids, e.g., α-aminoadipic acid (Aad), β-aminoadipic acid (βAad), α-aminobutyric acid (Abu), α-aminoisobutyric acid (Aib), β-alanine (βAla), 4-aminobutyric acid (4-Abu), 5-aminovaleric acid (5-Ava), 6-aminohexanoic acid (6-Ahx), 8-aminooctanoic acid (8-Aoc), 9-aminononanoic acid (9-Anc), 10-aminodecanoic acid (10-Adc), 12-aminododecanoic acid (12-Ado), α-aminosuberic acid (Asu), azetidine-2-carboxylic acid (Aze), β-cyclohexylalanine (Cha), aitrulline (Cit), dehydroalanine (Dha), γ-carboxyglutamic acid (Gla), α-cyclohexylglycine (Chg), propargylglycine (Pra), pyroglutamic acid (Glp), α-tert-butylglycine (Tle), 4-benzoylphenylalanine (Bpa), δ-hydroxylysine (Hyl), 4-hydroxyproline (Hyp), allo-isoleucine (alle), lanthionine (Lan), (1-naphthyl)alanine (1-Nal), (2-naphthyl)alanine (2-NaI), norleucine (Nle), norvaline (Nva), ornithine (Orn), phenylglycin (Phg), pipecolic acid (Pip), sarcosine (Sar), selenocysteine (Sec), statine (Sta), β-thienylalanine (Thi), 1,2,3,4-tetrahydroisochinoline-3-carboxylic acid (Tic), allo-threonine (aThr), thiazolidine-4-carboxylic acid (Thz), γ-aminobutyric acid (GABA), iso-cysteine (iso-Cys), diaminopropionic acid (Dpr), 2,4-diaminobutyric acid (Dab), 3,4-diaminobutyric acid (γβDab), biphenylalanine (Bip), phenylalanine substituted in para-position with —C₁-C₆ alkyl, -halide, —NH₂, —CO₂H or Phe(4-R) (wherein R═—C₁-C₆ alkyl, -halide, —NH₂, or —CO₂H); peptide nucleic acids (PNA, cf., P. E. Nielsen, Acc. Chem. Res., 32, 624-30);
or their N-alkylated analogues, such as their N-methylated analogues.

Cyclic amino acids may be proteinogenic or non-proteinogenic, such as Pro, Aze, Glp, Hyp, Pip, Tic and Thz.

For further examples and details reference can be made to, e.g., J. H. Jones, J. Peptide Sci., 2003, 9, 1-8 which is herein incorporated by reference.

As used hereinafter in the description of the invention and in the claims, the terms “non-proteinogenic amino acid” and “non-proteinogenic amino acid residue” also encompass derivatives of proteinogenic amino acids. For example, the side chain of a proteinogenic amino acid residue may be derivatized thereby rendering the proteinogenic amino acid residue “non-proteinogenic”. The same applies to derivatives of the C-terminus and/or the N-terminus of a proteinogenic amino acid residue terminating the amino acid sequence.

As used hereinafter in the description of the invention and in the claims, a proteinogenic amino acid residue is derived from a proteinogenic amino acid selected from the group consisting of Ala, Arg, Asn, Asp, Cys, Gln, Glu, Gly, His, Ile, Leu, Lys, Met, Phe, Pro, Ser, Thr, Trp, Tyr and Val either in L- or D-configuration; the second chiral center in Thr and Ile may have either R- or S-configuration. Therefore, for example, any posttranslational modification of an amino acid sequence, such as N-alkylation, which might naturally occur renders the corresponding modified amino acid residue “non-proteinogenic”, although in nature said amino acid residue is incorporated in a protein. Preferably modified amino acids are selected from N-alkylated amino acids, β-amino acids, γ-amino acids, lanthionines, dehydro amino acids, and amino acids with alkylated guanidine moieties.

As used hereinafter in the description of the invention and in the claims, the term “peptidomimetic” relates to molecules which are related to peptides, but with different properties. A peptidomimetic is a small protein-like chain designed to mimic a peptide. They typically arise from modification of an existing peptide in order to alter the molecule's properties. For example, they may arise from modifications to change the molecule's stability or biological activity. This can have a role in the development of drug-like compounds from existing peptides. These modifications involve changes to the peptide that will not occur naturally.

As used hereinafter in the description of the invention and in the claims, the term “peptide analogs”, by itself refers to synthetic or natural compounds which resemble naturally occurring peptides in structure and/or function.

As used hereinafter in the description of the invention and in the claims, the term “pharmaceutically acceptable salt” relates to salts of inorganic and organic acids, such as mineral acids, including, but not limited to, acids such as carbonic, nitric or sulfuric acid, or organic acids, including, but not limited to acids such as aliphatic, cycloaliphatic, aromatic, araliphatic, heterocyclic, carboxylic and sulphonic acids, examples of which are formic, acetic, trifluoroacetic, propionic, succinic, glycolic, gluconic, lactic, malic, fumaric, pyruvic, benzoic, anthranilic, mesylic, salicylic, phenylacetic, mandelic, embonic, methansulfonic, ethanesulfonic, benzenesulfonic, phantothenic, toluenesulfonic and sulfanilic acid.

If a chiral center or another form of an isomeric center is present in a compound having general chemical Formulae A, I, II, III or IV of the present invention, as given hereinafter, all forms of such isomers, including enantiomers and diastereoisomers, are intended to be covered herein. Compounds containing a chiral center may be used as a racemic mixture or as an enantiomerically enriched mixture, or the racemic mixture may be separated using well-known techniques and an individual enantiomer maybe used alone. In cases in which compounds have unsaturated carbon-carbon double bonds, both the cis-isomer and trans-isomers are within the scope of this invention. In cases in which compounds may exist in tautomeric forms, such as keto-enol tautomers, each tautomeric form is contemplated as being included within the scope of the present invention whether existing in equilibrium or predominantly in one form.

As used hereinafter in the description of the invention and in the claims, the term “oligonucleotide” shall have the following meaning: short sequences of nucleotides, typically with twenty or fewer bases. Examples are, but are not limited to, molecules named and cited in the book: “The aptamers handbook. Functional oligonuclides and their application” by Svenn Klussmann, Wiley-VCH, 2006. An example for such an oligonucleotide is TTA1 (J. Nucl Med., 2006, April, 47(4):668-78).

As used hereinafter in the description of the invention and in the claims, the term “aptamer” refers to an oligonucleotide, comprising from 4 to 100 nucleotides, wherein at least two single nucleotides are connected to each other via a phosphodiester linkage. Said aptamers have the ability to bind specifically to a target molecule (see, e.g., M Famulok, G Mayer, “Aptamers as Tools in Molecular Biology and Immunology”, in: “Combinatorial Chemistry in Biology, Current Topics in Microbiology and Immunology” (M Famulok, C H Wong, E L Winnacker, Eds.), Springer Verlag Heidelberg, 1999, Vol. 243, 123-136). There are many ways known to the skilled person of how to generate such aptamers that have specificity for a certain target molecule. An example is given in WO 01/09390 A, the disclosure of which is hereby incorporated by reference. Said aptamers may comprise substituted or non-substituted natural and non-natural nucleotides. Aptamers can be synthesized in vitro using, e.g., an automated synthesizer. Aptamers according to the present invention can be stabilized against nuclease degradation, e.g., by the substitution of the 2′-OH group versus a 2′-fluoro substituent of the ribose backbone of pyrimidine and versus 2′-O-methyl substituents in the purine nucleic acids. In addition, the 3′ end of an aptamer can be protected against exonuclease degradation by inverting the 3′ nucleotide to form a new 5′-OH group, with a 3′ to 3′ linkage to a penultimate base.

For the purpose of this invention, the term “nucleotide” refers to molecules comprising a nitrogen-containing base, a 5-carbon sugar, and one or more phosphate groups. Examples of said base comprise, but are not limited to, adenine, guanine, cytosine, uracil, and thymine. Also non-natural, substituted or non-substituted bases are included. Examples of 5-carbon sugar comprise, but are not limited to, D-ribose, and D-2-desoxyribose. Also other natural and non-natural, substituted or non-substituted 5-carbon sugars are included. Nucleotides as used in this invention may comprise from one to three phosphates.

As used hereinafter in the description of the invention and in the claims, the term “halogen” refers to F, Cl, Br and I.

If a chiral center or another form of an isomeric center is present in a compound, all forms of such isomers, including enantiomers and diastereoisomers, are intended to be covered herein. Compounds containing a chiral center may be used as a racemic mixture or as an enantiomerically enriched mixture, or the racemic mixture may be separated using well-known techniques and an individual enantiomer maybe used alone. In cases in which compounds have unsaturated carbon-carbon double bonds, both the cis-isomer and trans-isomers are within the scope of this invention. In cases in which compounds may exist in tautomeric forms, such as keto-enol tautomers, each tautomeric form is contemplated as being included within the scope of the present invention whether existing in equilibrium or predominantly in one form.

Abbreviations used throughout the specification are used within the following meanings:

| | |
|---|---|
| Ts | tosyl |
| Ns | nitrophenylsulfenyl |
| Cbz | carbobenzoxy |
| Bz | benzoyl |
| Bn | benzyl |
| Boc | tert-butoxycarbonyl |
| Fmoc | 9-fluorenylmethoxycarbonyl |
| Tr | triphenylmethyl |

The object of the present invention is solved as detailed below.

In a first aspect, the present invention provides novel compounds comprising an aziridine ring being appropriately activated for labelling purposes, wherein a targeting agent radical, either directly or via an appropriate linker, is attached either to the aziridine ring or to a fused five-membered carbocyclic or heterocyclic ring which is fused to the aziridine ring.

In a preferred first alternative according to the first aspect of the present invention, such compound may be represented by general chemical Formula I:

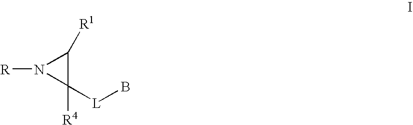

wherein
R represents Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, Ns, Cbz, Bz, Bn, Boc, Fmoc, methoxycarbonyl, ethoxycarbonyl, allyloxycarbonyl, Tr or acyl;
   wherein R² represents hydrogen, substituted or unsubstituted or linear or branched C₁-C₆ alkyl, cycloalkyl, heterocycloalkyl, aryl, heteroaryl, aralkyl or heteroaralkyl, OH, OR³, NH₂, NHR³, N(R³)₂, SH, SR³, halogen, NO₂, C(═O)R³, C(═O)OR³, C(═O)NHR³ or C(═O)(NR³)₂,
   R³ represents hydrogen, substituted or non-substituted, linear or branched C₁-C₆ alkyl, aryl, cycloalkyl, heterocycloalkyl, aryl, heteroaryl, aralkyl or heteroaralkyl;
R¹ and R⁴, independently, are selected from the group comprising hydrogen, substituted and non-substituted, linear and branched C₁-C₆ alkyl, cycloalkyl, heterocycloalkyl, aryl, heteroaryl, aralkyl and heteroaralkyl;
L represents a linker suitable for coupling with the targeting agent radical; and
B represents the targeting agent radical.

According to this first alternative, the invention further refers to pharmaceutically acceptable salts of an inorganic or organic acid thereof, hydrates, complexes, esters, amides, solvates and prodrugs of the compounds having general chemical Formula I.

In a preferred embodiment of this first alternative, R may be Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, or Ns;
wherein R² represents hydrogen, substituted or non-substituted, linear or branched C₁-C₆ alkyl, OH, OR³, NH₂, NHR³, N(R³)₂, SH, SR³, Cl, Br, I, NO₂, C(═O)R³, C(═O)OR³, C(═O)NHR³, C(═O)N(R³)₂;
R³ represents hydrogen or substituted or non-substituted, linear or branched C₁-C₆ alkyl.

In a more preferred embodiment of this first alternative, R may be 2,4,6-triisopropyl-phenyl-sulfonyl, 3,4-dimethoxy-phenyl-sulfonyl, unsubstituted phenyl-sulfonyl or phenyl-sulfonyl being substituted with 1-5 R² moieties;
wherein R² represents hydrogen, substituted or non-substituted, linear or branched C₁-C₆ alkyl or OR³, wherein R³ represents substituted or non-substituted, linear or branched C₁-C₆ alkyl.

In a preferred embodiment of this first alternative, R¹ and R⁴, independently, may be selected from the group comprising hydrogen and substituted and non-substituted, linear and branched C₁-C₆ alkyl.

In a more preferred embodiment of this first alternative, R¹ and R⁴ may represent hydrogen.

In this preferred first alternative according to the first aspect, which may also be defined using the following alternative general chemical Formula A, which is congruent to Formula I:

RG-L₁-B₁—Y-E A

wherein
RG is a group or groups of atoms or a reactive moiety attached to L₁ that can form an adduct with a fluorine isotope, to provide a chemically and biologically stable bond,
L₁ is a moiety group or bond to which the reactive group (RG) is attached,
B₁ is a functional group or a chain containing functional group connecting linker to spacer,
Y is a bond or a spacer,
E is a biomolecule.
The compound having general chemical Formula I herein above may be defined using general chemical Formula A above, if RG is N-substituted aziridine:

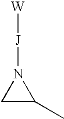

wherein J is SO₂, CO,
with the proviso that if J is SO₂, then W is unsubstituted or substituted phenyl, NH₂, NHR³, N(R³)₂, linear or branched C₁-C₆ alkyl, aryl, heteroaryl, wherein substitution of the phenyl ring is independently or in combinations selected from linear or branched C₁-C₆ alkyl,
R³ is C₁-C₆ alkyl or aralkyl,
Further, if J is CO, then W is unsubstituted or substituted phenyl, benzyloxy, fluorenylmethyl, methoxy, ethoxy, or allyloxy,
wherein substitution of the phenyl ring is independently or in combinations selected from linear or branched C₁-C₆ alkyl.

Referring to general chemical Formula A, in a more preferred embodiment, RG is selected from the group comprising N-benzenesulfonylaziridinyl, N-p-toluenesulfonylaziridinyl, N-2,4,6-triisopropylsulfonylaziridinyl, N-3,4-dimethoxy-phenylsulfonylaziridinyl. More preferably, RG may be N-benzenesulfonylaziridinyl,p-toluenesulfonylaziridinyl or N-2,4,6-triisopropylsulfonylaziridinyl.

Further referring to general chemical Formula A, in a more preferred embodiment, L₁ may be bond or linear or branched C₁-C₆ alkyl. Even more preferably, L₁ may be a bond.

Further, referring to general chemical Formula A, in a preferred embodiment, —B₁— may be selected from the group comprising a bond, —C(═O)—, —(CH₂)_{d}—C(═O)—, —SO—, —C≡C—C(═O)—, —[CH₂]ₘ-D-[CH₂]—C(═O), —[CH₂]ₘ-D-[CH₂]ₙ—SO₂—, —C(═O)—O—, —NR¹⁰—, —O—, —(S)ₚ—, —C(═O)NR¹²—, —C(═S)NR¹²—, —C(═S)O—, C₁-C₆ cycloalkyl, alkenyl, heterocycloalkyl, unsubstituted or substituted aryl or unsubstituted or substituted heteroaryl, aralkyl, heteroaralkyl, alkylenoxy, arylenoxy, aralkoxy, —SO₂NR¹³—, —NR¹³SO₂—, —NR¹³C(═O)O—, —NR¹³C(═O)NR¹²—, —NH—NH— and —NH—O—,

wherein d is an integer from 1 to 6,
m and n, independently, can be any integer from 0 to 5;
D represents a bond, —S—, —O— or —NR⁹—,
wherein R⁹ represents hydrogen, C₁-C₁₀ alkyl, aryl, heteroaryl, or aralkyl,
p can be any integer of from 1 to 3;
R¹⁰ and R¹², independently, represent hydrogen, C₁-C₁₀ alkyl, aryl, heteroaryl or aralkyl, and
R¹³ represents hydrogen, substituted or unsubstituted, linear or branched C₁-C₆ alkyl, aryl, cycloalkyl, heterocycloalkyl, aryl, heteroaryl, aralkyl, or heteroaralkyl.

Further, referring to general chemical Formula A, more preferably, —B₁— is preferably selected from —C(═O)— and —C≡C—C(═O)— and even more preferably —B₁— is —C(═O)—.

In this alternative definition, relative to the compound having general chemical Formula I, RG corresponds to the moiety

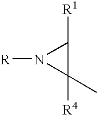

the group L₁-B₁ corresponds to L (linker) and the group Y-E corresponds to B (targeting agent), wherein E is a biomolecule.

Preferred compounds of the present invention are:
1-(Toluene-4-sulfonyl)-aziridine-2-carboxylic acid-Gly-Val-βAla-Phe-Gly-amide
1-(2,4,6-Triisopropyl-benzenesulfonyl)-aziridine-2-carboxylic-Gly-Val-βAla-Phe-Gly-amide
1-(2,4,6-Triisopropyl-benzenesulfonyl)-aziridine-2-carboxylic acid [(3-{3-[(2R,4S,5R)-4-(1-methoxy-cyclohexyloxy)-5-(1-methoxy-cyclohexyloxymethyl)-tetrahydro-furan-2-yl]-5-methyl-2,6-dioxo-3,6-dihydro-2H-pyrimidin-1-yl}-propylcarbamoyl)-methyl]-amide

In a preferred second alternative according to the first aspect of the present invention, such compound is represented by general chemical Formula II:

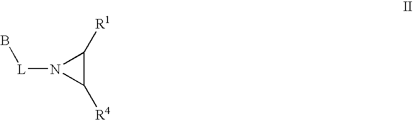

wherein
R¹ and R⁴, independently, are selected from the group comprising hydrogen, substituted and non-substituted, linear and branched C₁-C₆ alkyl, cycloalkyl, heterocycloalkyl, aryl, heteroaryl, aralkyl and heteroaralkyl;
L represents a linker suitable for coupling with the targeting agent radical and for appropriate activation of the aziridine ring; and
B represents the targeting agent radical.

According to this second alternative, the invention further refers to pharmaceutically acceptable salts of an inorganic or organic acid thereof, hydrates, complexes, esters, amides, solvates and prodrugs of the compounds having general chemical Formula II.

In a preferred embodiment of this second alternative R¹ and R⁴, independently, may be selected from the group comprising hydrogen and substituted and non-substituted, linear and branched C₁-C₆ alkyl.

In a preferred third alternative according to the first aspect of the present invention, such compound is represented by general chemical Formula III:

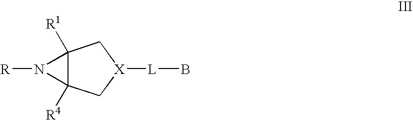

wherein
R represents Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, Ns, Cbz, Bz, Bn, Boc, Fmoc, methoxycarbonyl, ethoxycarbonyl, allyloxycarbonyl, Tr or acyl;
   wherein R² represents hydrogen, substituted or non-substituted, linear or branched C₁-C₆ alkyl, cycloalkyl, heterocycloalkyl, aryl, heteroaryl, aralkyl, or heteroaralkyl, OH, OR³, NH₂, NHR³, N(R³)₂, SH, SR³, Cl, Br, I, NO₂, C(═O)R³, C(═O)OR³, C(═O)NHR³ or C(═O)N(R³)₂;
   wherein R³ represents hydrogen, substituted or non-substituted, linear or branched C₁-C₆ alkyl, aryl, cycloalkyl, heterocycloalkyl, aryl, heteroaryl, aralkyl, or heteroaralkyl;
R¹ and R⁴, independently, are selected from the group comprising hydrogen, substituted and non-substituted, linear and branched C₁-C₆ alkyl, cycloalkyl, heterocycloalkyl, aryl, heteroaryl, aralkyl and heteroaralkyl;
X represents N or C substituted by a hydrogen;
L represents a linker suitable for coupling with the targeting agent radical; and
B represents the targeting agent radical.

According to this third alternative, the invention further refers to pharmaceutically acceptable salts of an inorganic or organic acid thereof, hydrates, complexes, esters, amides, solvates and prodrugs of the compounds having general chemical Formula III.

In a preferred embodiment of this third alternative R may be Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, or Ns;
wherein R² represents hydrogen, substituted or non-substituted, linear or branched C₁-C₆ alkyl, OH, OR³, NH₂, NHR³, N(R³)₂, SH, SR³, Cl, Br, I, NO₂, C(═O)R³, C(═O)OR³, C(═O)NHR³, C(═O)N(R³)₂;
wherein R³ represents hydrogen, substituted or non-substituted, linear or branched C₁-C₆ alkyl or aryl;

R¹ and R⁴, independently, may be selected from the group comprising hydrogen and substituted and non-substituted, linear and branched C₁-C₆ alkyl;

X may represent N or C substituted by a hydrogen;

In a further preferred embodiment R may be Ts, 2,4,6-triisopropyl-phenyl-sulfonyl, 3,4-dimethoxy-phenyl-sulfonyl, unsubstituted phenyl-sulfonyl or phenyl-sulfonyl being substituted with 1-5 R² moieties;
wherein R² represents hydrogen, substituted or non-substituted, linear or branched C₁-C₆ alkyl, OR³, SR³, Cl, Br, I, C(═O)R³, C(═O)OR³, C(═O)NHR³ or C(═O)N(R³)₂, wherein R³ represents hydrogen, substituted or non-substituted, linear or branched C₁-C₆ alkyl or aryl;
   R¹ and R⁴, independently, may be selected from the group comprising hydrogen and substituted and non-substituted, linear and branched C₁-C₆ alkyl; and
   X may represent N.

In all alternatives, the linker -L- is preferably selected from the group consisting of substituted and non-substituted, linear and branched C₁-C₆ alkyl, cycloalkyl, alkenyl, heterocycloalkyl, unsubstituted or substituted aryl, unsubstituted or substituted heteroaryl, aralkyl, heteroaralkyl, alkyloxy, aryloxy, aralkoxy, —C(═O)—, —C(═O)O—, —C(═O)NH—, —C(═O)N—(CH₂)ₙ—C(═O)—, —C(═O)—(CH₂)ₙ—C(═O)—, —SO₂—, —SO₂NR³—, —NR³SO₂—, —NR³C(═O)O—, —NR³C(═O)NR³—, —NR³—, —NH—NH—, —NH—O—, —(CH₂)ₙ—C(═O)—NR³—CH₂—C(═O)—, —SO₂— (unsubstituted or substituted aryl)-(CH₂)ₙ—C(═O)—,

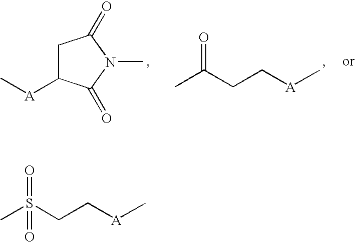

wherein n may be from 1 to 3, -A- may represent —S— or —NR³—;
wherein R³ represents hydrogen, substituted or non-substituted, linear or branched C₁-C₆ alkyl, cycloalkyl, heterocycloalkyl, aryl, heteroaryl, aralkyl or heteroaralkyl.

The linker -L- may more preferably be selected from the group comprising linear and branched C₁-C₆ alkyl, -(substituted and unsubstituted, linear and branched C₁-C₆ alkyl)-C(═O)—, —C(═O)—, —C(═O)NH—, —C(═O)N—(CH₂)ₙ—C(═O)— or —C(═O)—(CH₂)ₙ—C(═O)— with n=1-3.

Further, in all alternatives, the targeting agent radical B may preferably comprise a biomolecule selected from the group comprising peptides, small molecules and oligonucleotides. The biomolecules may also be peptidomimetics.

If the biomolecule is a small molecule, the linker -L- is preferably not —C(═O)—. Thus, in such case, -L- may preferably:
be selected from the group consisting of substituted and non-substituted, linear and branched C₁-C₆ alkyl, cycloalkyl, alkenyl, heterocycloalkyl, unsubstituted or substituted aryl, unsubstituted or substituted heteroaryl, aralkyl, heteroaralkyl, alkyloxy, aryloxy, aralkoxy, —C(═O)O—, —C(═O)NH—, —C(═O)N—(CH₂)ₙ—C(═O)— and —C(═O)—(CH₂)ₙ—C(═O)—, —SO₂—, —SO₂NR³—, —NR³SO₂—, —NR³C(═O)O—, —NR³C(═O)NR³—, —NR³—, —NH—NH—, —NH—O—(CH₂)ₙ—C(═O)—NR³—CH₂—C(═O)—, —SO₂— (unsubstituted or substituted aryl)-(CH₂)ₙ—C(═O)—,

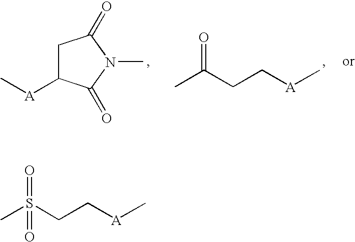

wherein n may be from 1 to 3,
-A- may represent —S— or —NR³—;
wherein R³ represents hydrogen, substituted or non-substituted, linear or branched C₁-C₆ alkyl, cycloalkyl, heterocycloalkyl, aryl, heteroaryl, aralkyl or heteroaralkyl;
   or even more preferably:
be selected from the group comprising linear and branched C₁-C₆ alkyl, -(substituted and unsubstituted, linear and branched C₁-C₆ alkyl)-C(═O)—, —C(═O)NH—, —C(═O)N—(CH₂)ₙ—C(═O)— or —C(═O)—(CH₂)ₙ—C(═O)— with n=1-3.

The targeting agent radical B comprises a biomolecule E to which latter may be optionally linked a reacting moiety Y which serves the linking between the biomolecule and the rest of the compound and which may be, e.g., —NR′, —(CH₂)ₙ—NR′—, —(CH₂)ₙ—O— or —(CH₂)ₙ—S—, wherein R′ is hydrogen or alkyl and n is an integer from 1 to 6. Thus, B is Y-E, wherein Y is bond or a spacer.

In a more preferred embodiment Y is selected a spacer selected from natural or unnatural amino acid sequence or non-amino acid group.

More preferably, Y may be an amino acid sequence with two (2) to twenty (20) amino acid residues.

More preferably, Y may be Arg-Ser, Arg-Ava, Lys(Me)2-β-ala, Lys(Me)2-ser, Arg-β-ala, Ser-Ser, Ser-Thr, Arg-Thr, S-alkylcysteine, Cysteic acid, thioalkylcysteine (S—S-Alkyl) or

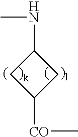

wherein k and l is 0-4.

More preferably, Y may be a non-amino acid moiety selected from NH—(CH₂)ₚ—C(═O)

wherein p being an integer from 2 to 10,
NH—(CH₂—CH₂—O)_{q}—CH₂—CH₂—C(═O) wherein q being an integer from 0 to 5 —NH-cycloalkyl-CO— wherein cycloalkyl is selected from C₅-C₈ cycloalkyl, more preferably C₆ atom cycloalkyl, and
—NH-heterocycloalkyl-(CH₂)ᵥ—CO— wherein heterocycloalkyl is selected from C₅-C₈ heterocycloalkyl containing carbon atoms and 1, 2, 3 or 4 oxygen, nitrogen or sulfur heteroatoms more preferably 1 to 2 heteroatom even more preferably 1 heteroatom and v is an integer of from 1 to 4, more preferably v is an integer of from 1 to 2.

E is a biomolecule. The biomolecule E is preferably selected from the group comprising peptides, peptidomimetics, small molecules and oligonucleotides.

As used hereinafter in the description of the invention and in the claims, the terms “targeting agent” and “biomolecules” are directed to compounds or moieties that target or direct the radionuclide attached to them to a specific site in a biological system. A targeting agent or biomolecule can be any compound or chemical entity that binds to or accumulates at a target site in a mammalian body, i.e., the compound localizes to a greater extent at the target site than to surrounding tissue.

Small molecules effective for targeting certain sites in a biological system can be used as the biomolecule E. Smaller organic molecules may be “small chemical entities”. As used in this application, the term “small chemical entity” shall have the following meaning: A small chemical entity is a compound that has a molecular mass of from 200 to 800 or of from 150 to 700, more preferably of from 200 to 700, more preferably of from 250 to 700, even more preferably of from 300 to 700, even more preferably of from 350 to 700 and most preferably of from 400 to 700. A small chemical entity as used herein may further contain at least one aromatic or heteroaromatic ring and/or may also have a primary and/or secondary amine, a thiol or hydroxyl group coupled via L to the rest of the molecule in the compounds of general chemical Formulae I, II and III. Such targeting moieties are known in the art, so are methods for preparing them.

The small molecule targeting agents/biomolecules may preferably be selected from those described in the following references: P. L. Jager, M. A. Korte, M. N. Lub-de Hooge, A. van Waarde, K. P. Koopmans, P. J. Perik and E. G. E. de Vries, Cancer Imaging, (2005) 5, 27-32; W. D. Heiss and K. Herholz, J. Nucl Med., (2006) 47(2), 302-312; and T. Higuchi and M. Schwaiger, Curr. Cardiol. Rep., (2006) 8(2), 131-138. More specifically examples of small molecule targeting agents/biomolecules are listed hereinafter:

| Name | Abbr. | target |
|---|---|---|
| 18F-2b-Carbomethoxy-3b-(4- | CFT | DAT (dopamine transporter) |
| fluorophenyl)tropane | | |
| 18F-Fluoroethylspiperone | FESP | D2 (dopamine 2 receptor), 5- |
| | | HT₂ (5-hydroxytryptamine receptor) |
| 18F-Fallypride | | D2 (dopamine 2 receptor) |
| 18F-Altanserin | | 5-HT2A receptor |
| 18F-Cyclofoxy | | Opioid receptors |
| 18F-CPFPX | | Adenosine A1 receptor |
| Batimastat | | MMP |
| Fatty acids and analogues | | |
| Choline analogues | | |
| (metabolism) | | |
| Flumazenil | | Benzodiazepine receptors |
| Raclopride | | D2 receptors |
| Dihydrotestosteron and | | AR |
| analogues | | |
| Tamoxifen and analogues | | |
| Deoxyglucose | | |
| Thymidine | | Proliferation marker- thymidine |
| | | kinase |
| DOPA | | |
| Benzazepines | | D₁ antagonists |
| N-methyl spiperone and | | dopamine receptors |
| derivatives thereof | | |
| Benzamide raclopride; | | D₂ receptors |
| benzamide derivatives, e.g., | | |
| fallopride, iodo benzamide; | | |
| clozapine, quietapine | | |
| Nomifensine, substituted | | DAT |
| analogs of cocaine, e.g., | | |
| tropane type derivatives of | | |
| cocaine, methyl phenidate | | |
| 2β-Carboxymethoxy-3β-(4- | CIT | DAT |
| iodophenyl)tropane | | |
| | CIT-FE, CIT-FM | DAT |
| Altanserin, setoperon, | | 5-HT_{2A} |
| ketanserin | | |
| | McN5652, 403U76 derivative | 5-HTT |
| | ADAM, DASP, MADAM | |
| Acetylcholine analogues | MP3A, MP4A, PMP; QNB, | acetylcholine receptors |
| | TKB, NMPB, | |
| Scopolamine, benztropine | | acetylcholine receptors |
| Flumazenil | | GABA receptor |
| | RO-15-4513, FDG | GABA receptor |
| | PK-11195 | benzodiazepine receptor |
| Xanthine analogues | CPFPX, MPDX | adenosine receptor |
| Carfentanyl, diprenorphine | | opoid receptor |

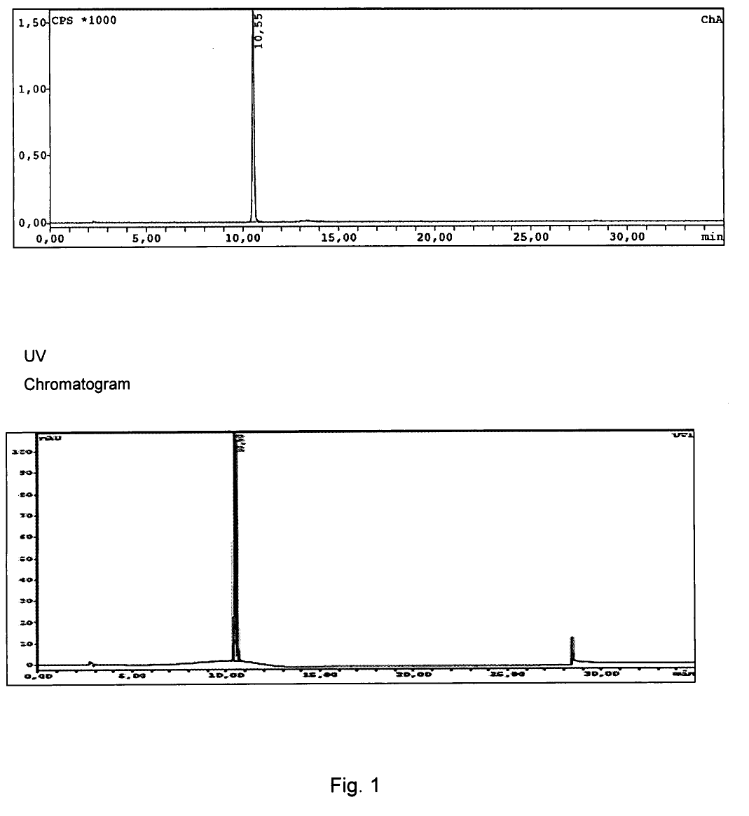

Further various small molecule targeting agents/biomolecules and the targets thereof are given in Table 1 in W. D. Heiss and K. Herholz, ibid. and in FIG. 1 in T. Higuchi, M. Schwaiger, ibid.

Further preferred biomolecules are sugars, oligosaccharides, polysaccharides, aminoacids, nucleic acids, nucleotides, nucleosides, oligonucleotides, proteins, peptides, peptidomimetics, antibodies, aptamers, lipids, hormones (steroid and nonsteroid), neurotransmitters, drugs (synthetic or natural), receptor agonists and antagonists, dendrimers, fullerenes, virus particles and other targeting molecules/biomolecules (e.g., cancer targeting molecules).

Further, the biomolecule E may be a peptide. E may be a peptide comprising from 2 to 100 amino acids, more preferably 4 to 100 amino acids.

In a further preferred embodiment of the present invention, the biomolecule may be a peptide which is selected from the group comprising somatostatin and derivatives thereof and related peptides, somatostatin receptor specific peptides, neuropeptide Y and derivatives thereof and related peptides, neuropeptide Y₁ and the analogs thereof, bombesin and derivatives thereof and related peptides, gastrin, gastrin releasing peptide and the derivatives thereof and related peptides, epidermal growth factor (EGF of various origin), insulin growth factor (IGF) and IGF-1, integrins (α₃β₁, αᵥβ₃, αᵥβ₅, αIIb₃), LHRH agonists and antagonists, transforming growth factors, particularly TGF-α; angiotensin; cholecystokinin receptor peptides, cholecystokinin (CCK) and the analogs thereof; neurotensin and the analogs thereof, thyrotropin releasing hormone, pituitary adenylate cyclase activating peptide (PACAP) and the related peptides thereof, chemokines, substrates and inhibitors for cell surface matrix metalloproteinase, prolactin and the analogs thereof, tumor necrosis factor, interleukins (IL-1, IL-2, IL-4 or IL-6), interferons, vasoactive intestinal peptide (VIP) and the related peptides thereof. Such peptides comprise from 4 to 100 amino acids, wherein the amino acids are selected from natural and non-natural amino acids and also comprise modified natural and non-natural amino acids.

In a more preferred embodiment of the present invention, the biomolecule may be selected from the group comprising bombesin and bombesin analogs, preferably those having the sequences listed herein below, somatostatin and somatostatin analogs, preferably those having the sequences listed herein below, neuropeptide Y₁ and the analogs thereof, preferably those having the sequences listed herein below, vasoactive intestinal peptide (VIP) and the analogs thereof.

In a more preferred embodiment of the present invention, the biomolecule may be selected from the group comprising bombesin, somatostatin, neuropeptide Y₁. Vasoactive intestinal peptide (VIP) and the analogs thereof.

In an even more preferred embodiment of the present invention, the biomolecule E may be bombesin, somatostatin or neuropeptide Y₁ or an analog thereof.

In an even more preferred embodiment of the present invention, the biomolecule may be bombesin or an analog thereof.

Bombesin is a fourteen amino acid peptide that is an analog of human gastrin releasing peptide (GRP) that binds with high specificity to human GRP receptors present in prostate tumor, breast tumor and metastasis. In an even more preferred embodiment of the present invention, the biomolecule E comprises bombesin analogs having sequence III or IV:
AA₁-AA₂-AA₃-AA₄-AA₅-AA₆-AA₇-AA₈-NT₁T₂ (type A) III, with
   T₁=T₂=H, T₁=H, T₂=OH, T₁=CH₃, T₂=OH
   AA₁=Gln, Asn, Phe(4-CO—NH₂)
   AA₂=Trp, D-Trp
   AA₃=Ala, Ser, Val
   AA₄=Val, Ser. Thr
   AA₅=Gly, (N-Me)Gly
   AA₆=His, His(3-Me), (N-Me)His, (N-Me)His(3-Me)
   AA₇=Sta, Statine analogs and isomers, 4-Am, 5-MeHpA, 4-Am, 5-MeHxA and γ-substituted aminoacids
   AA₈=Leu, Cpa, Cba, CpnA, Cha, t-buGly, tBuAla, Met, Nle, iso-Bu-Gly
AA₁-AA₂-AA₃-AA₄-AA₅-AA₆-AA₇-AA₈-NT₁T₂ (type B) IV, with:
   T₁=T₂=H, T₁=H, T₂=OH, T₁=CH₃, T₂=OH
   AA₁=Gln, Asn, Phe(4-CO—NH₂)
   AA₂=Trp, D-Trp
   AA₃=Ala, Ser, Val
   AA₄=Val, Ser. Thr
   AA₅=βAla, β²- and β³-amino acids as shown herein after

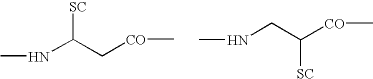

wherein SC represents side chain found in proteinogenic amino acids and homologs of proteinogenic amino acids,
AA₆=His, His(3-Me), (N-Me)His, (N-Me)His(3-Me)
AA₇=Phe, Tha, NaI,
AA₈=Leu, Cpa, Cba, CpnA, Cha, t-buGly, tBuAla, Met, Nle, iso-Bu-Gly.

Therefore, in an even more preferred embodiment of the present invention the biomolecule may be selected from the group comprising bombesin analogs having sequence III or IV.

In a more preferred embodiment, bombesin analogs have the following sequences:

| | |
|---|---|
| Seq ID | E |

| | |
|---|---|
| Seq ID 1 | Gln-Trp-Ala-Val-NMeGly-His-Sta-Leu-NH₂ |
| Seq ID 2 | Gln-Trp-Ala-Val-Gly-His(Me)-Sta-Leu-NH₂ |
| Seq ID 3 | Gln-Trp-Ala-Val-NMeGly-His(3Me)-Sta- |
| | Leu-NH₂ |
| Seq ID 4 | Gln-Trp-Ala-Val-Gly-His(3Me)-Sta-Leu- |
| | NH₂ |
| Seq ID 7 | Gln-Trp-Ala-Val-NMeGly-His(3Me)-Sta- |
| | Cpa-NH₂ |
| Seq ID 8 | Gln-Trp-Ala-Val-Gly-His(3Me)-4-Am,5- |
| | MeHpA-Leu-NH₂ |
| Seq ID 12 | Gln-Trp-Ala-Val-Gly-His(3Me)-4-Am,5- |
| | MeHpA-Leu-NH₂ |
| Seq ID 17 | Gln-Trp-Ala-Val-Gly-His-4-Am,5-MeHpA- |
| | Leu-NH₂ |
| Seq ID 23 | Gln-Trp-Ala-Val-NMeGly-His(3Me)-4-Am,5- |
| | MeHpA-Cpa-NH₂ |
| Seq ID 27 | Gln-Trp-Ala-Val-NMeGly-His-FA02010-Cpa- |
| | NH₂ |
| Seq ID 28 | Gln-Trp-Ala-Val-NMeGly-His-4-Am,5- |
| | MeHpA-tbuGly-NH₂ |
| Seq ID 30 | Gln-Trp-Ala-Val-NMeGly-His(3Me)-Sta- |
| | tBuGly-NH₂ |
| Seq ID 32 | Gln-Trp-Ala-Val-NMeGly-His(3Me)-4-Am,5- |
| | MeHpA-Leu-NH₂ |
| Seq ID 33 | Gln-DTrp-Ala-Val-Gly-His-4-Am,5-MeHpA- |
| | tbuGly-NH₂ |
| Seq ID 34 | Gln-DTrp-Ala-Val-Gly-His-4-Am-5-MeHxA- |
| | Cpa-NH₂ |
| Seq ID 35 | Gln-Trp-Ala-Val-NMeGly-His(3Me)-Sta- |
| | Cpa-NH₂ |
| Seq ID 36 | Gln-DTrp-Ala-Val-Gly-His-Sta-tbuAla-NH₂ |
| Seq ID 42 | Gln-Trp-Ala-Val-Gly-His(3Me)-Sta-Cpa- |
| | NH₂ |
| Seq ID 43 | Gln-Trp-Ala-Val-Gly-His(3Me)-Sta- |
| | tBuGly-NH₂ |
| Seq ID 46 | Gln-Trp-Ala-Val-Gly-His(3Me)-4-Am,5- |
| | MeHpA-Leu-NH₂ |
| Seq ID 48 | Gln-Trp-Ala-Val-Gly-His(3Me)-4-Am,5- |
| | MeHpA-Leu-NH₂ |
| Seq ID 49 | Gln-Trp-Ala-Val-Gly-NMeHis-4-Am,5- |
| | MeHpA-Cpa-NH₂ |
| Seq ID 49 | Gln-Trp-Ala-Val-Gly-NMeHis(3Me)-4-Am,5- |
| | MeHpA-Leu-NH₂ |
| Seq ID 50 | Gln-Trp-Ala-Val-Gly-NMeHis-4-Am,5- |
| | MeHpA-Leu-NH₂ |
| Seq ID 51 | Gln-Trp-Ala-Val-NMeGly-Hls-AHMHxA-Leu- |
| | NH₂ |
| Seq ID 52 | Gln-Trp-Ala-Val-βAla-NMeHis-Tha-Cpa-NH₂ |
| Seq ID 53 | Gln-Trp-Ala-Val-βAla-NMeHis-Phe-Cpa-NH₂ |
| Seq ID 54 | Gln-Trp-Ala-Val-βAla-NMeHis-Phe-Leu-NH₂ |
| Seq ID 55 | Gln-Trp-Ala-Val-βAla-DHis-Phe-Leu-NH₂ |
| Seq ID 56 | Gln-Trp-Ala-Val-βAla-His-I3hLeu-Leu-NH₂ |
| Seq ID 57 | Gln-Trp-Ala-Val-βAla-His-13h Ile-Leu- |
| | NH₂ |
| Seq ID 58 | Gln-Trp-Ala-Val-βAla-His-I3hLeu-tbuGly- |
| | NH₂ |
| Seq ID 59 | Gln-Trp-Ala-Val-βAla-His(3Me)-Phe-Tha- |
| | NH₂ |
| Seq ID 60 | Gln-Trp-Ala-Val-βAla-His(3Me)-Phe-Nle- |
| | NH₂ |
| Seq ID 61 | Gln-Trp-Ala-Val-βAla-NMeHis-Phe-tbuGly- |
| | NH₂ |
| Seq ID 62 | Gln-Trp-Ala-Val-βAla-NMeHis-Tha-tbuGly- |
| | NH₂ |
| Seq ID 63 | Gln-Trp-Ala-Val-βAla-His(3Me)-Tha- |
| | tbuGly-NH₂ |
| Seq ID 64 | Gln-Trp-Ala-Val-βAla-His(3Me)-Phe-Cpa- |
| | NH₂ |
| Seq ID 65 | Gln-Trp-Ala-NMeVal-βAla-His-Phe-Leu-NH₂ |
| Seq ID 66 | Gln-Trp-Ala-Val-βAla-His-NMePhe-Leu-NH₂ |
| Seq ID 67 | Gln-DTrp-Ala-Val-βAla-His-Phe-Leu-NH₂ |
| Seq ID 68 | Gln-Trp-DAla-Val-βAla-His-Phe-Leu-NH₂ |
| Seq ID 69 | Gln-Trp-Ala-DVal-βAla-His-Phe-Leu-NH₂ |
| Seq ID 70 | Gln-Trp-Ala-Val-βAla-His-DPhe-Leu-NH₂ |
| Seq ID 71 | Gln-Trp-Ala-Val-βAla-His-βhIle-tbuGly- |
| | NH₂ |
| Seq ID 72 | Gln-Trp-Ala-Val-NMeGly-His-4-Am,5- |
| | MeHpA-Cpa-NH₂ |
| Seq ID 73 | Gln-Trp-Ala-Val-NMeGly-His-Sta-Cpa-NH₂ |
| Seq ID 74 | Gln-Trp-Ala-Val-NMeGly-His-Sta-tbuAla- |
| | NH₂ |
| Seq ID 75 | Gln-Trp-Ala-Val-NMeGly-His-4-Am,5- |
| | MeHpA-tbuAla-NH₂ |
| Seq ID 77 | Gln-Trp-Ala-Val-His(Me)-Sta-Leu-NH₂ |
| Seq ID 82 | Gln-Trp-Ala-Val-Gly-His(3Me)-FA4-Am,5- |
| | MeHpA-Leu-NH₂ |
| Seq ID 90 | Gln-Trp-Ala-Val-Gly-His(3Me)-4-Am,5- |
| | MeHpA-Leu-NH₂ |
| Seq ID 91 | Gln-Trp-Ala-Val-Gly-His-4-Am,5-MeHpA- |
| | Leu-NH₂ |
| Seq ID 101 | Gln-Trp-Ala-Val-Gly-His(3Me)-4-Am-5- |
| | MeHpA-4-amino-5-methylheptanoic acid- |
| | Leu-NH₂ |
| Seq ID 102 | Gln-Trp-Ala-Val-NMeGly-His(3Me)-4-Am-5- |
| | MeHpA-4-amino-5-methylheptanoic acid- |
| | Cpa-NH₂ |

More preferably a bombesin analog is additionally labeled with a fluorine atom (F) wherein fluorine atom (F) is selected from ¹⁸F or ¹⁹F. More preferably the bombesin analog is radiolabeled with ¹⁸F. The bombesin analog is preferably radiolabeled using the radiofluorination method of the present invention.

The above bombesin analogs that bind specifically to human GRP receptors present in prostate tumor, breast tumor and metastasis, may be part of the compound having general chemical Formula I, in that they form the biomolecule, wherein the biomolecule may optionally be linked to a reacting moiety Z which serves the linking between the biomolecule and the rest of the compound of the invention (Formulae I, II), e.g., —NR′, —NR′—(CH₂)ₙ—, —O—(CH₂)ₙ— or —S—(CH₂)ₙ—, wherein R′ is hydrogen or alkyl and n is an integer from 1 to 6. The bombesin analogs may be peptides having sequences from Seq ID 1 to Seq ID 102 and preferably may have one of them. More preferably a bombesin analog is additionally radiolabelled with a fluorine isotope (F) wherein F is ¹⁸F or ¹⁹F. More preferably the bombesin analog is radiolabelled using the radiofluorination method of the present invention.

In a more preferred embodiment, somatostatin analogs have the following sequences:

Seq ID 104----c[Lys-(NMe)Phe-1Nal-D-Trp-Lys-Thr]
Seq ID 105----c[Dpr-Met-(NMe)Phe-Tyr-D-Trp-Lys]

In a more preferred embodiment, neuropeptide Y₁ analogs have the following sequences:

Seq ID 106-DCys-Leu-Ile-Thr-Arg-Cys-Arg-Tyr-NH₂
Seq ID 107-DCys-Leu-Ile-Val-Arg-Cys-Arg-Tyr-NH₂
( indicates disulfide bridge)

In a more preferred embodiment the peptide is tetrapeptide having any one of the following sequences:
valyl-β-alanyl-phenylalanyl-glycine amide
valyl-β-alanyl-histidyl(π-Me)-glycine amide

In a further preferred embodiment the targeting agent B may be selected from the group comprising oligonucleotides comprising from 4 to 100 nucleotides. Preferred oligonucleotide is TTA1 (see experimental part).

In a further preferred embodiment of the present invention, the biomolecule E may comprise a combination of any of the aforementioned bioactive molecules suitable to bind to a target site together with a reacting moiety which serves the linking between the bioactive molecule and the rest of the compound of the invention (Formulae I, II, III), e.g., —NR′, —NR′—(CH₂)ₙ—, —O—(CH₂)ₙ— or —S—(CH₂)ₙ—, wherein R′ is hydrogen or alkyl and n is an integer from 1 to 6.

According to a second aspect, the present invention is directed to a method of preparing the novel compounds, preferably the compounds having any one of general chemical Formulae I, II and III, by reacting a suitable precursor molecule with the targeting agent or a precursor thereof.

A third aspect of the present invention relates to novel fluorinated compounds and to pharmaceutically acceptable salts of inorganic or organic acids thereof, to hydrates, complexes, esters, amides, solvates and prodrugs thereof.

In a first alternative of this third aspect, the present invention relates to a compound obtainable by a ring opening fluorination reaction of the aziridine ring of one of the novel compounds of the first aspect of the present invention, more preferably of any one of the compounds having general chemical Formulae I, II and III. In this first alternative, the present invention also relates to pharmaceutically acceptable salts, hydrates, complexes, esters, amides, solvates and prodrugs thereof.

In a second alternative of this third aspect, the present invention relates to a fluorinated compound, having any one of general chemical Formulae I-F-A and I-F-B:

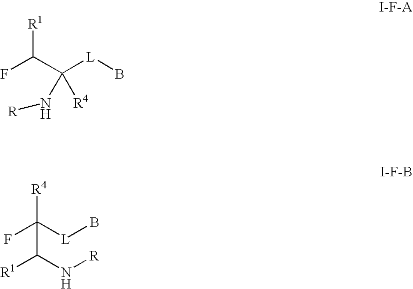

wherein R, R¹, R⁴, L and B have the meanings as given herein above; F is fluorine isotope as defined herein above.

According to this second alternative, the invention further refers to pharmaceutically acceptable salts of an inorganic or organic acid thereof, hydrates, complexes, esters, amides, solvates and prodrugs of the compounds having any one of general chemical Formulae I-F-A and I-F-B.

In this preferred second alternative according to the second aspect, the present invention relates to a radiopharmaceutical labelled with fluorine having general chemical Formula B

F-L₂-B₂—Y-E B

wherein
F is fluorine isotope
L₂ is a moiety group or bond to which F is attached
B₂ is a functional group or chain containing functional group connecting L₂ with the spacer Y
Y is a bond or spacer
E is a biomolecule.

In a preferred embodiment F is ¹⁸F or ¹⁹F.

More preferably, of F is ¹⁸F then the radiopharmaceutical labelled with fluorine has general chemical Formula B-A.

[18]F-L₂-B₂—Y-E B-A

More preferably, if F is ¹⁹F then the pharmaceutical labelled with fluorine has general chemical Formula B-B.

[19]F-L₂-B₂—Y-E B-B

wherein L₂ is α-(substituted)amino-ethyl to which F is attached at β-position, J and W are defined as herein above:

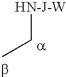

B₂ of general chemical Formula B is identical to B₁ of general chemical Formula A and preferred embodiment.

Y of general chemical Formula B is identical to Y of general chemical Formula A and preferred embodiment.

E of general chemical Formula B is identical to E of general chemical Formula A and preferred embodiment.

In a third alternative of this third aspect, the present invention relates to a fluorinated compound, having any one of general chemical Formulae II-F-A and II-F-B:

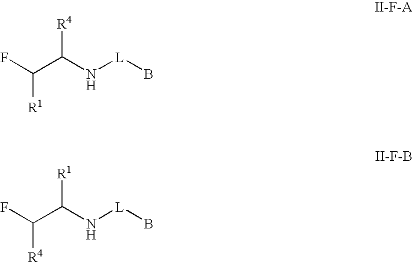

wherein R, R¹, R⁴, L and B have the meanings as given herein above; F is fluorine isotope as defined herein above.

According to this third alternative, the invention further refers to pharmaceutically acceptable salts of an inorganic or organic acid thereof, hydrates, complexes, esters, amides, solvates and prodrugs of the compounds having any one of general chemical Formulae II-F-A and II-F-B.

In a fourth alternative of this third aspect, the present invention relates to a fluorinated compound, having any one of general chemical Formulae III-F-A and III-F-B:

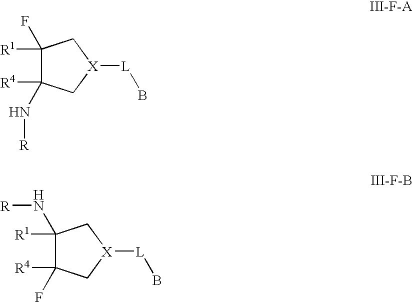

wherein R, R¹, R⁴, L and B have the meanings as given herein above; F is fluorine isotope as defined herein above.

According to this fourth alternative, the invention further refers to pharmaceutically acceptable salts of an inorganic or organic acid thereof, hydrates, complexes, esters, amides, solvates and prodrugs of the compounds having any one of general chemical Formulae III-F-A and III-F-B.

In a fifth aspect, the present invention relates to a composition comprising a compound or a pharmaceutically acceptable salt of an inorganic or organic acid thereof, a hydrate, complex, ester, amide, solvate or prodrug thereof according to the first aspect of the present invention, e.g., a compound having any one of general chemical Formulae I, II and III, and a fluorinated compound or a pharmaceutically acceptable salt of an inorganic or organic acid thereof, a hydrate, complex, ester, amide, solvate or prodrug thereof according to the third aspect of the present invention, e.g., a compound having any one of general chemical Formulae I-F-A, I-FI-B, II-F-A, II-F-B, III-F-A and III-F-B. The composition further comprises a pharmaceutically acceptable carrier, diluent, excipient or adjuvant.

In a sixth aspect, the present invention relates to a kit comprising a sealed vial containing a predetermined quantity of a general chemical Formulae I, II and III of the first aspect along with an acceptable carrier, diluent, excipient or adjuvant for the manufacture of compounds of the third aspect.

In a further aspect, the present invention is directed to a kit comprising any of the fluorinated compounds as defined hereinabove or a composition comprising the same, e.g., in powder form, and a container containing an appropriate solvent for preparing a solution of the compound or composition for administration to an animal, including a human.

In a seventh aspect, the present invention is directed to the use of any fluorinated compound, as defined hereinabove, or respective composition or kit, for diagnostic imaging, in particular positron emission tomography. The use most preferably serves the imaging of tumors, imaging of inflammatory and/or neurodegenerative diseases, such as multiple sclerosis of Alzheimer's disease, or imaging of angiogenesis-associates diseases, such as growth of solid tumors, and rheumatoid arthritis.

Further, the present invention in this aspect thereof is directed to a fluorinated compound labelled with ¹⁸F isotope, for use as medicament, more preferably for use as diagnostic imaging agent and more preferably for use as imaging agent for positron emission tomography. In another variation of this aspect, the present invention also relates to fluorinated compounds, which are more preferably labelled with ¹⁹F isotope and which have general chemical Formulae I-F-A, I-F-B, II-F-A, II-F-B, III-F-A and III-F-B for use in biological assays and chromatographic identification. More preferably, the invention relates to the use of compound having any one of general chemical Formulae I, II and III for the manufacture of a compound having any one of general chemical Formulae I-F-A, I-F-B, II-F-A, II-F-B, III-F-A or III-F-B as a measurement agent.

In an eighth aspect, the present invention furthermore relates to a method of imaging diseases, said method comprising introducing into a patient a detectable quantity of a labelled compound having general chemical Formula I-F-A, I-F-B, II-F-A, II-F-B, III-F-A or III-F-B as defined herein above or of a pharmaceutically acceptable salt of an inorganic or organic acid thereof, a hydrate, complex, ester, amide, solvate and prodrug thereof and imaging patient.

The compounds of this invention are useful for the imaging of a variety of cancers including but not limited to carcinoma such as bladder, breast, colon, kidney, liver, lung, including small cell lung cancer, esophagus, gall-bladder, ovary, pancreas, stomach, cervix, thyroid, prostate and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer and Karposi's sarcoma.

Most preferably, the use is not only for imaging of tumors, but also for imaging of inflammatory and/or neurodegenerative diseases, such as multiple sclerosis or Alzheimer's disease, or imaging of angiogenesis-associated diseases, such as growth of solid tumors, and rheumatoid arthritis.

The radioactively labeled compounds according to Formulae I-F-A, I-F-B, II-F-A, II-F-B, III-F-A and III-F-B provided by the invention may be administered intravenously in any pharmaceutically acceptable carrier, e.g., conventional medium such as an aqueous saline medium, or in blood plasma medium, as a pharmaceutical composition for intravenous injection. Such medium may also contain conventional pharmaceutical materials such as, for example, pharmaceutically acceptable salts to adjust the osmotic pressure, buffers, preservatives and the like. Among the preferred media are normal saline and plasma. Suitable pharmaceutical acceptable carriers are known to the person skilled in the art. In this regard reference can be made to e.g., Remington's Practice of Pharmacy, 11^{th} ed. and in J. of. Pharmaceutical Science & Technology, Vol. 52, No. 5, September-October, p. 238-311 see table page 240 to 311, both publication include herein by reference.

The concentration of the fluorinated compound having general chemical Formulae I-F-A, I-F-B, II-F-A, II-F-B, III-F-A and III-F-B and the pharmaceutically acceptable carrier, for example, in an aqueous medium, varies with the particular field of use. A sufficient amount is present in the pharmaceutically acceptable carrier when satisfactory visualization of the imaging target (e.g., a tumor) is achievable.

In accordance with the invention, the radiolabelled compounds having general chemical Formulae I-F-A, I-F-B, II-F-A, II-F-B, III-F-A and III-F-B either as a neutral composition or as a salt with a pharmaceutically acceptable counter-ion are administered in a single unit injectable dose. Any of the common carriers known to those with skill in the art, such as sterile saline solution or plasma, can be utilized after radiolabelling for preparing the injectable solution to diagnostically image various organs, tumors and the like in accordance with the invention. Generally, the unit dose to be administered for a diagnostic agent has a radioactivity of about 0.1 mCi to about 100 mCi, preferably 1 mCi to 20 mCi. For a radiotherapeutic agent, the radioactivity of the therapeutic unit dose is about 10 mCi to 700 mCi, preferably 50 mCi to 400 mCi. The solution to be injected at unit dosage is from about 0.01 ml to about 30 ml. For diagnostic purposes after intravenous administration, imaging of the organ or tumor in vivo can take place in a matter of a few minutes. However, imaging takes place, if desired, in hours or even longer, after injecting into patients. In most instances, a sufficient amount of the administered dose will accumulate in the area to be imaged within about 0.1 of an hour to permit the taking of scintigraphic images. Any conventional method of scintigraphic imaging for diagnostic purposes can be utilized in accordance with this invention.

Thus, embodiments of this invention include methods involving the ¹⁸F fluorination of compounds ready for use as imaging agents. The compounds subjected to fluorination, may already include a targeting agent for imaging purposes. Preferred embodiments of this invention involve the formation of a precursor molecule, which may include a targeting agent, prior to fluorination with ¹⁸F, being the last step in the process prior to preparation of the compound for administration to an animal, in particular a human.

The use of aziridines described herein facilitates the process. Thus, a desired PET imaging agent is proposed starting from an aziridine which is then subjected to ¹⁸F fluorination.

Substituents on such aziridines include linking groups or reactive groups designed for subsequent addition of a targeting agent. Linking groups may include aliphatic or aromatic molecules and readily form a bond to a selected, appropriately functionalized targeting agent. A variety of such groups is known in the art. These include carboxylic acids, carboxylic acid chlorides and active esters, sulfonic acids, sulfonyl-chlorides, amines, hydroxides, thiols etc. on either side.

Contemplated herein are also groups which provide for ionic, hydrophobic and other non-convalent bonds between the aziridine ring and the targeting agent.

In a fourth aspect, the present invention is directed to a method of preparing such compounds by reacting one of the novel aziridine compounds according to the first aspect as defined hereinabove with an appropriate fluorinated agent.

Appropriate conditions comprise but are not limited to, those radiofluorination reactions which are carried out, for example, in a typical reaction vessel (e.g., Wheaton vial) being known to those skilled in the art or in a microreactor. The reaction can be heated by typical methods, e.g., using an oil bath, a heating block or microwave.

Preferably, said fluorinating agent may be K¹⁸F, H¹⁸F, KH¹⁸F₂ or a tetraalkyl ammonium salt of ¹⁸F⁻, most preferably K¹⁸F.

A solvent may be used, which can be DMF, DMSO, MeCN, DMA, DMAA, preferably DMSO. The solvents can also be a mixture of solvents as indicated above.

The radiofluorination reactions can be carried out in dimethylformamide with potassium carbonate as base and “kryptofix” as crown-ether. But also other solvents can be used which are well known to experts. In a preferred embodiment, the fluorination agent is 4,7,13,16,21,24-Hexaoxa-1,10-diazabicyclo[8.8.8]-hexacosane K18F (crownether salt Kryptofix K18F), K¹⁸F, H¹⁸F, KH¹⁸F₂ or tetraalkylammonium salt of ¹⁸F. More preferably, the fluorination agent is K¹⁸F, H¹⁸F, or KH¹⁸F₂.

The possible conditions mentioned include, but are not limited to: dimethylsulfoxid and acetonitrile as solvent and tetraalkyl ammonium and tetraalkyl phosphonium carbonate as base. Water and/or alcohol can be involved in such a reaction as co-solvent. The radiofluorination reactions are conducted for 1 to 45 minutes. Preferred reaction times are 3 to 40 minutes. Further preferred reaction times are 5 to 30 min.

This novel condition comprises the use of inorganic acid and/or organic acid in the ¹⁸F radiolabelling, reaction. Preferably organic acids are used in the ¹⁸F radiolabelling, reaction. More preferably aliphatic, cycloaliphatic, aromatic, araliphatic, heterocyclic carboxylic and sulphonic acids are used in the ¹⁸F radiolabelling, reaction. Most preferably aliphatic carboxylic acids are used, including but not limited to propionic acid, acetic acid and formic acid.

The method may preferably be run under a reaction temperature of 100° C. or less, most preferably 80° C. or less.

In a preferred method of preparing a compound having any one of general chemical Formulae I-F-A, I-F-B, II-F-A, II-F-B, III-F-A and III-F-B, the step of radiofluorination of a compound having any one of general chemical Formulae I, II and III is carried out at a temperature at or below 90° C., more preferably at a temperature in a range of from 10° C. to 90° C., even more preferably at a reaction temperature from room temperature to 80° C., even more preferably at a temperature in a range of from 10° C. to 70° C., even more preferably at a temperature in a range of from 30° C. to 60° C., even more preferably at a temperature in a range of from 45 to 55° C. and most preferably at a temperature at 50° C.

A new method is warranted in which the final product is prepared in a single step from the precursor. Only a single purification step is optionally carried out thereby the preparation can be accomplished in a short time (considering the half-life of ¹⁸F). In a typical prosthetic group preparation, very often temperatures of 100° C. and above are employed. The invention provides methods to accomplish the preparation at temperatures (80° C. or below) that preserve the biological properties of the final product.

### EXAMPLE FOR LABELLING

### First Example

¹⁸F-fluoride (up to 40 GBq) was azeotropically dried in the presence of Kryptofix 222 (5 mg in 1.5 ml MeCN) and cesium carbonate (2.3 mg in 0.5 ml water) by heating under a stream of nitrogen at 110-120° C. for 20-30 minutes. During this time 3×1 ml MeCN were added and evaporated. After drying, a solution of the precursor (2 mg) in 150 μl DMSO was added. The reaction vessel was sealed and heated at 50-70° C. for 5-15 mins to effect labelling. The reaction was cooled to room temperature and diluted with water (2.7 ml). The crude reaction mixture was analyzed using an analytical HPLC. The product was obtained by preparative radio HPLC to give the desired ¹⁸F labelled peptide.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The entire disclosure[s] of all applications, patents and publications, cited herein are incorporated by reference herein.

The following examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### General Method for the Preparation of Compounds

The targeting agent radical portion, preferably peptide portion, of the molecule part E-Z-Y— can be conveniently prepared according generally established techniques known in the art of peptide synthesis, such as solid-phase peptide synthesis. They are amenable Fmoc-solid phase peptide synthesis, employing alternate protection and deprotection. These methods are well documented in peptide literature. (Reference: “Fmoc Solid Phase Peptide Synthesis” A practical approach”, Edited by W. C. Chan and P. D. White, Oxford University Press 2000) (For Abbreviations see Descriptions).

### EXAMPLES

Examples of the preparation/synthesis of precursor compounds are shown below and are illustrative for some of the embodiments of the invention described herein. These examples should not be considered to limit the spirit or scope of the invention in any way. The aziridine moiety of these precursors can easily be fluorinated, such as fluorinated with ¹⁸F. Cold (¹⁹F) compounds were prepared and are necessary as references, e.g., for HPLC analysis of labelled products.

### Methods of Preparing Compounds Having General Chemical Formulae I, II and III

Scheme 1 shows a possible way for synthesis of compounds having general chemical Formula I.

Compounds having general chemical Formula I can be synthesised starting with commercial aziridines 1 or from α-amino alcohols via mesylation or tosylation of the alcohol and nucleophilic substitution towards the formation of aziridines 1 (not shown). Depending on the substitution pattern on the aziridine, it might be necessary to perform the first steps of appropriate functionalisation with an inert protecting group, such as trityl. If the substitution pattern leads to a more stable aziridine, electron deficient activation groups as needed for fluorination, respectively, might be included straight from the beginning of the synthetic sequence. In the procedure shown here, the aziridine is protected first with a trityl group followed by saponification of the methyl ester 2. The resulting acid 3 can be converted to an active ester 4 followed by treatment with glycine or directly be coupled with glycine to yield the aziridine derivative 5 with an extended linker. This is necessary if n=0 as aziridines directly substituted with a carboxylate functionality are less stable than aziridines substituted with amides. This linker extension is not necessary if n>0. In the next step the trityl protection is cleaved and several other groups (6), preferably substituted aryl sulfonyl groups, can be introduced to activate the aziridine towards nucleophilic substitution (fluorination). Saponification to 7 leads to building blocks which can be added to targeting agents to give labelling precursors 8.

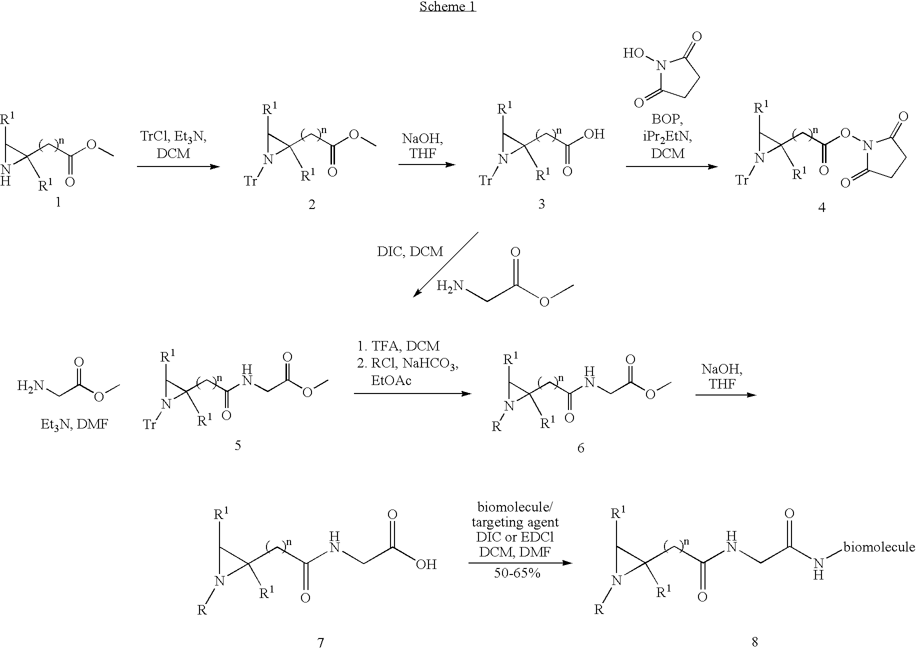

Scheme 2 shows a possible way of synthesis of compounds according having general chemical Formula II.

Compounds having general chemical Formula II can be synthesised starting with appropriate substituted aryl derivatives 13 by introducing a chlorosulfonyl group towards 14 followed by the addition of commercially available neat aziridine to give the substituted aziridine 15. Saponification leads to building blocks 16 which can be added to targeting agents to give labelling precursors 17.

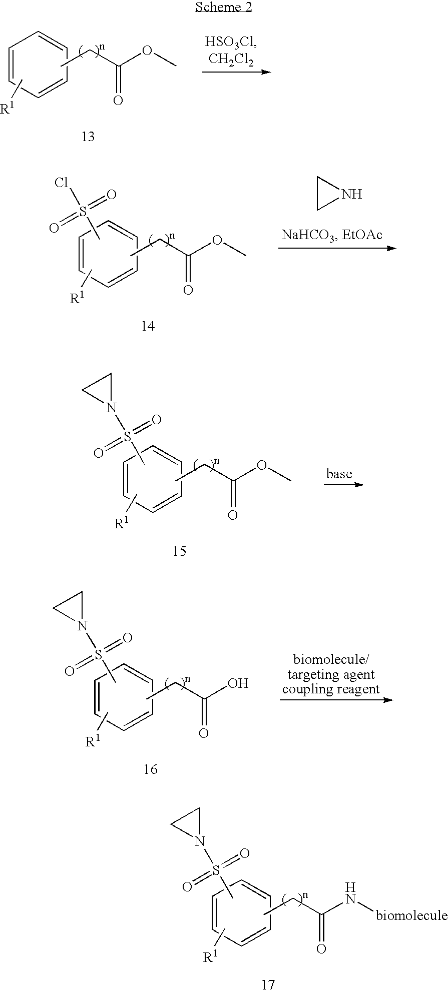

Scheme 3 shows a possible way of synthesis of compounds having general chemical Formula III.

Compounds having general chemical Formula III can be synthesised starting with the reaction of a dihydro pyrrole 20 and methyl 4-chloro-4-oxybutyrate 21 which leads to the substituted dihydro pyrrole 22. The following steps as epoxidation (23), opening of the epoxide with azide (24), tosylation of the resulting alcohol (25), Staudinger reduction of the azide followed by substitution of the tosylate (26) are used to generate the desired aziridine 26. Different types of activating groups R, preferably substituted aryl sulfonyl groups can be introduced to give 27. Saponification leads to building blocks 28 which can be added to targeting agents directly or via an active ester 29 to give labelling precursors 30.

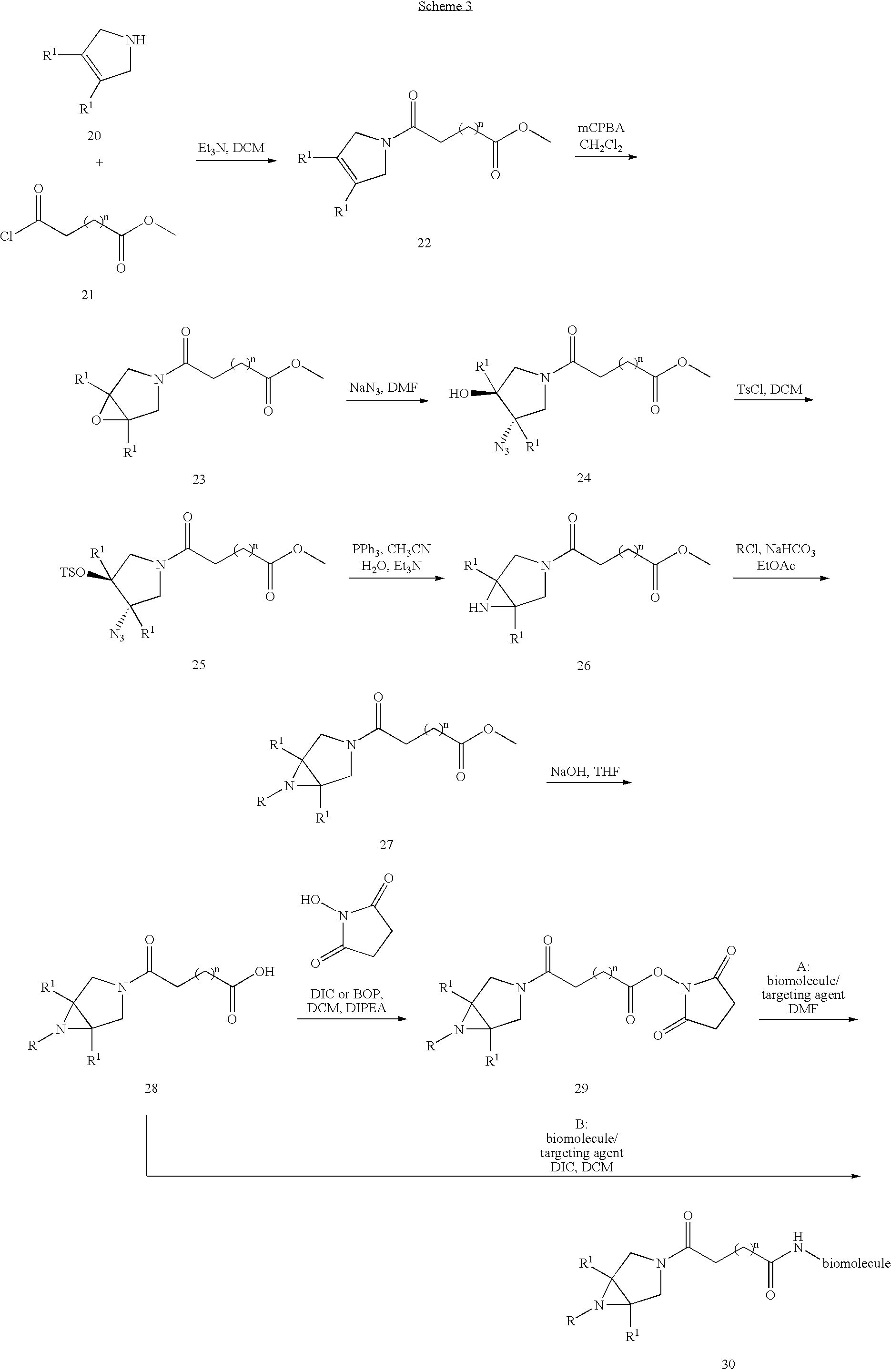

Experimental details can be seen from the experimental part hereinafter.

The fluorination reaction leading to labelled derivatives, as typical examples of fluorination reactions of all such different types of aziridine compounds is shown in Scheme 4.

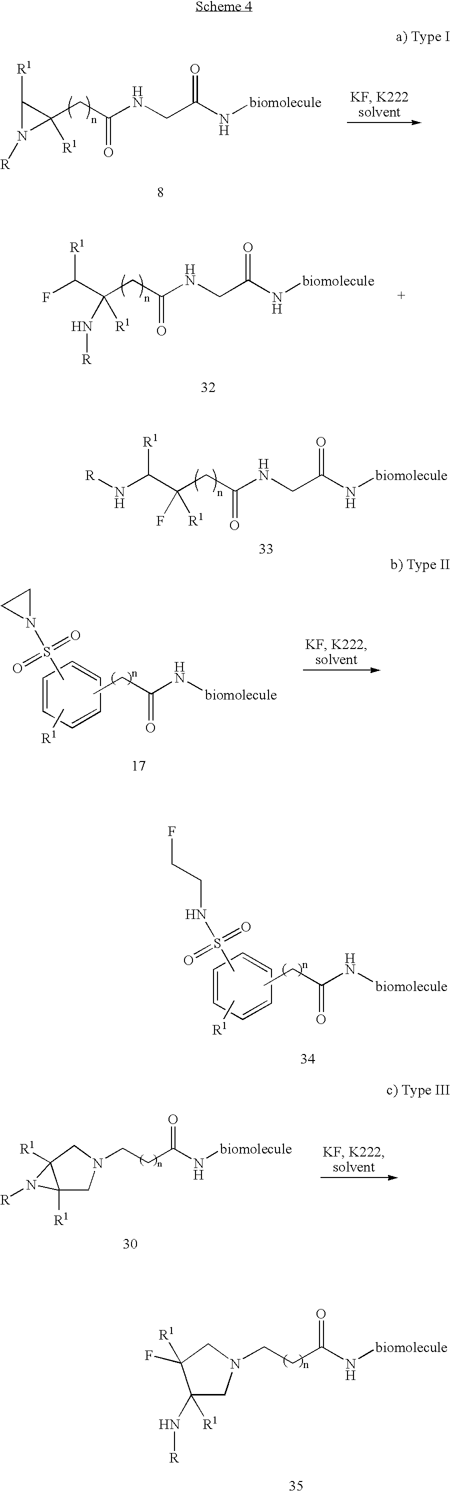

### EXPERIMENTAL PART

### Example 1

### Preparation of Compounds Having General Chemical Formula I and Corresponding Model Compounds

Preparation According to Scheme 1 with n=0.

### Preparation of 1-Trityl-aziridine-2-carboxylic acid methyl ester 2a

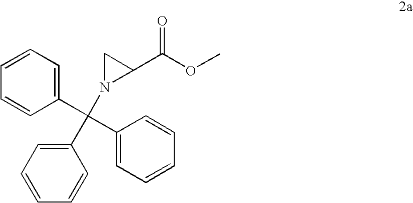

3 g (29.6 mmol) aziridine 1a was solved in 50 ml dichloromethane, cooled down to 0° C. followed by the addition of 6.17 ml (44.51 mmol) triethylamine and 9.93 g (35.61 mmol) trityl chloride. The reaction mixture was stirred at room temperature for 2 h and concentrated. The residue was purified by chromatography on silica gel to give 9.96 g (98%) of 2a.

¹H-NMR (CDCl₃): δ=7.41 (m, 6H), 7.30-7.17 (m, 9H), 3.77 (s, 3H), 2.26 (dd, 1H), 1.89 (dd, 1H), 1.42 (dd, 1H) ppm.

### Preparation of 1-Trityl-aziridine-2-carboxylic acid 3a

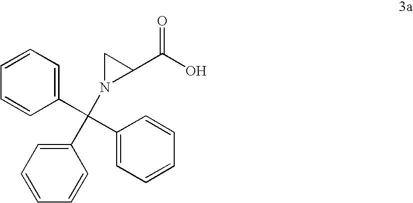

7.45 g (21.69 mmol) 2a were solved in 55 ml tetrahydrofurane, cooled down to OC and treated with 34.7 ml (34.71 mmol) 1 N sodium hydroxide solution. The reaction mixture was stirred overnight at room temperature, concentrated and the residue was purified by chromatography on silica gel to give 6.91 g (97%) of 3a.

¹H-NMR (MeOD): δ=7.45 (m, 6H), 7.30-7.17 (m, 9H), 2.16 (dd, 1H), 1.78 (dd, 1H), 1.40 (dd, 1H) ppm.

### Preparation of 1-Trityl-aziridine-2-carboxylic acid-2,5-dioxo-pyrrolidin-1-yl ester 4a

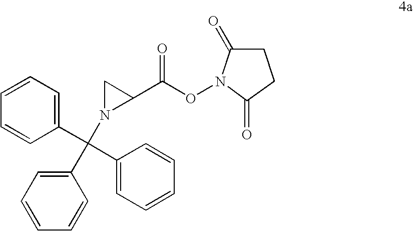

910 mg (2.76 mmol) 3a were solved in dichloromethane, 1.34 g (3.04 mmol) BOP and 318 mg (2.76 mmol) N-hydroxysuccinimide were added and the solution was cooled down to 0° C. Then 0.76 ml (4.42 mmol) ethyl diisopropylamine was added slowly and the reaction was stirred overnight at room temperature. The reaction mixture was diluted with dichloromethane, washed with 10% citric acid and brine, dried over sodium sulfate and concentrated. The residue was purified by chromatography on silica gel to give 760 mg (64%) of 4a.

¹H-NMR (MeOD): δ=7.45 (m, 6H), 7.30-7.17 (m, 9H), 2.84 (s, 4H), 2.44 (m, 1H), 2.09 (dd, 1H), 1.60 (dd, 1H) ppm.

### Preparation of {[1-(Trityl)-aziridine-2-carbonyl]-amino}acetic acid methyl ester 5a

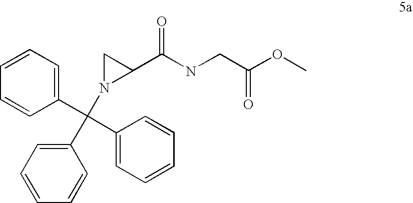

218 mg (1.74 mmol) glycin methylester hydrochloride was solved in DMF and treated with 0.36 ml (2.6 mmol) triethyl amine. After 30 min at room temperature 740 mg (1.74 mmol) 4a was added. The reaction mixture was stirred for 2 h at 50° C. and then concentrated. The residue was purified by chromatography on silica gel to give 550 (79%) of 5a.

¹H-NMR (CDCl₃): δ=7.45 (m, 6H), 7.30-7.17 (m, 9H), 4.22 (dd, 1H), 4.10 (dd, 1H), 3.81 (s, 3H), 2.05 (m, 2H), 1.50 (dd, 1H) ppm.

### Preparation of {[1-(Toluene-4-sulfonyl)-aziridine-2-carbonyl]-amino}acetic acid methyl ester 6aa

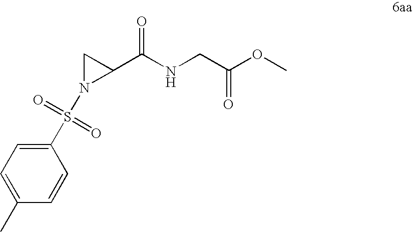

2.3 g (5.74 mmol) 5a was solved in 95 ml chloroform, cooled down to 0° C. and titrated with trifluoro acetic acid until complete conversion. The mixture was neutralized with saturated sodium bicarbonate solution and concentrated. The residue was suspended in 95 ml ethyl acetate and 95 ml saturated sodium bicarbonate solution followed by (11.49 mmol) sulfonic acid chloride. The reaction mixture was stirred overnight at room temperature. The phases were separated, the aqueous phase was extracted with ethyl acetate and the combined organic phases were dried over sodium sulphate and concentrated. The residue was purified by chromatography on silica gel to give (21-47%) of 6aa.

¹H-NMR (MeOD): δ=7.83 (d, 2H), 7.45 (d, 2H), 3.89 (s, 2H), 3.67 (s, 3H), 3.30 (d, 1H), 2.76 (d, 1H), 2.50 (d, 1H), 2.44 (s, 3H) ppm.

### Preparation of {[1-(2,4,6-Triisopropyl-benzenesulfonyl)-aziridine-2-carbonyl]-amino}acetic acid methyl ester 6ab

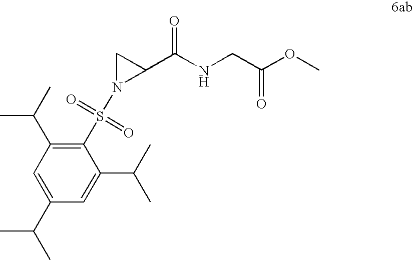

This compound was prepared in an analogous way to 6aa.

¹H-NMR (MeOD): δ=7.33 (s, 2H), 4.33 (sept, 2H), 3.98 (d, 2H), 3.73 (s, 3H), 3.43 (dd, 1H), 2.98 (sept, 1H), 2.87 (d, 1H), 2.60 (d, 1H), 1.32-1.28 (m, 18H) ppm.

### Preparation of {[1-(3,4-Dimethoxy-benzenesulfonyl)-aziridine-2-carbonyl]-amino}acetic acid methyl ester 6ac

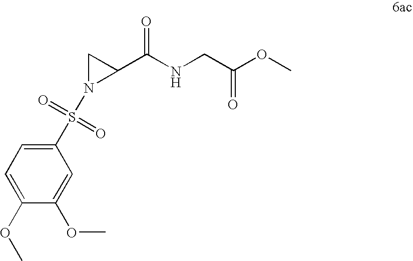

This compound was prepared in an analogous way to 6aa.

¹H-NMR (CDCl₃): δ=7.56 (dd, 1H), 7.41 (d, 1H), 7.00 (d, 1H), 6.62 (bt, 1H), 4.03 (dd, 1H), 3.97 (s, 3H), 3.92 (dd, 1H), 3.73 (s, 3H), 3.28 (dd, 1H), 2.83 (d, 1H), 2.46 (d, 1H) ppm.

### Preparation of {[1-(Toluene-4-sulfonyl)-aziridine-2-carbonyl]-amino}acetic acid 7aa

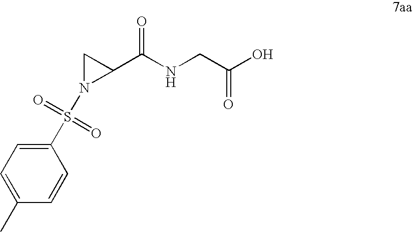

(1.18 mmol) 6aa was solved in 15 ml tetrahydrofurane, cooled down to 0° C. and treated with 0.71 ml (1.42 mmol) 2N sodium hydroxide solution. The reaction mixture was stirred at room temperature for 2 h and concentrated. The residue was taken up in water, carefully neutralized with citric acid and extracted with ethyl acetate. The combined organic phases were washed with brine, dried over sodium sulphate, filtrated and concentrated. The product 7aa (90-97%) was used without further purification.

¹H-NMR (MeOD): δ=7.83 (d, 2H), 7.44 (d, 2H), 3.86 (s, 2H), 3.30 (d, 1H), 2.76 (d, 1H), 2.51 (d, 1H), 2.44 (s, 3H) ppm.

### Preparation of {[1-(2,4,6-Triisopropyl-benzenesulfonyl)-aziridine-2-carbonyl]-amino}acetic acid 7ab

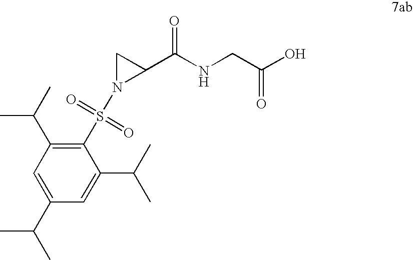

This compound was prepared in an analogous way to 7aa.

¹H-NMR (MeOD): δ=7.27 (s, 2H), 4.27 (sept, 2H), 3.90 (d, 2H), 3.99 (dd, 1H), 2.93 (sept, 1H), 2.78 (d, 1H), 2.55 (d, 1H), 1.30-1.23 (m, 18H) ppm.

### Preparation of {[1-(3,4-Dimethoxy-benzenesulfonyl)-aziridine-2-carbonyl]-amino}acetic acid 7ac

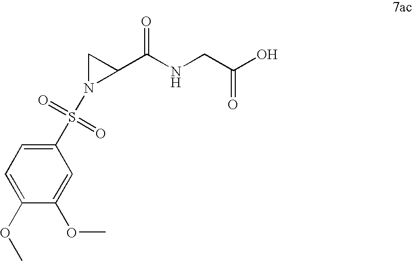

This compound was prepared in an analogous way to 7aa.

¹H-NMR (CDCl₃): δ=7.56 (dd, 1H), 7.39 (d, 1H), 6.99 (d, 1H), 6.78 (bt, 1H), 4.09 (dd, 1H), 3.96 (s, 3H), 3.94 (dd, 1H), 3.95 (s, 3H), 3.32 (dd, 1H), 2.80 (d, 1H), 2.46 (d, 1H) ppm.

### Preparation of 1-(Toluene-4-sulfonyl)-aziridine-2-carboxylic acid-Gly-Val-βAla-Phe-Gly-amide 8aaa

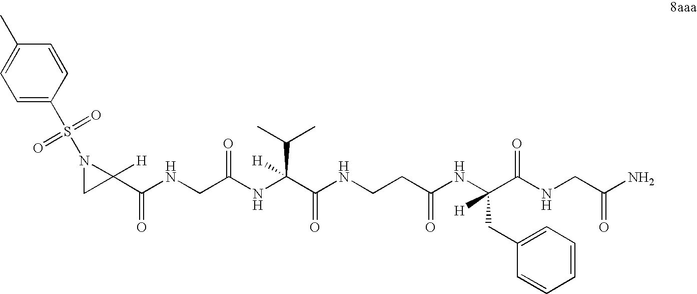

0.1 mmol resin bound di- or tetrapeptide, swollen in DMF was filtered and added to a solution of 0.3 mmol 7aa, 113.7 mg (0.3 mmol) HBTU and 104.5 μl (0.6 mmol) diisopropyl ethyl amine in 1.5 ml DMF. The mixture was shaken for 4 h, filtered and the remaining resin was washed with DMF and dichloromethane and dried under vacuum. Then the resin was treated with 1.5 ml of a mixture containing 85% TFA, 5% water, 5% phenol and 5% triisopropyl silane for 2 h, filtered followed by precipitation of the product in 20 ml MTBE. The precipitate was purified by HPLC to give 7-23% 8aaa.

HPLC-MS (ES+): m/z (%)=672 (100).

### Preparation of 1-(2,4,6-Tri isopropyl-benzenesulfonyl)-aziridine-2-carboxylic-Gly-Val-βAla-Phe-Gly-amide 8aba

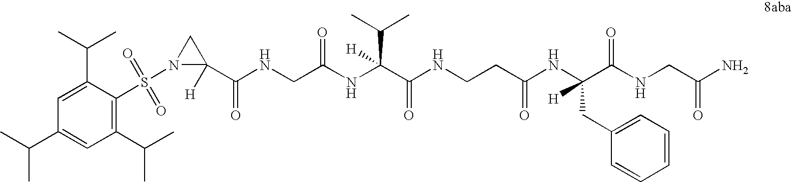

This compound was prepared in an analogous way to 8aaa starting from 7ab.

HPLC-MS (ES+): m/z (%)=784 (100).

### Preparation of 1-(2,4,6-Tri isopropyl-benzenesulfonyl)-aziridine-2-carboxylic acid [(3-{3-[(2R,4S,5R)-4-(1-methoxy-cyclohexyloxy)-5-(1-methoxy-cyclohexyloxymethyl)-tetrahydro-furan-2-yl]-5-methyl-2,6-dioxo-3,6-dihydro-2H-pyrimidin-1-yl}-propylcarbamoyl)-methyl]-amide 8abb

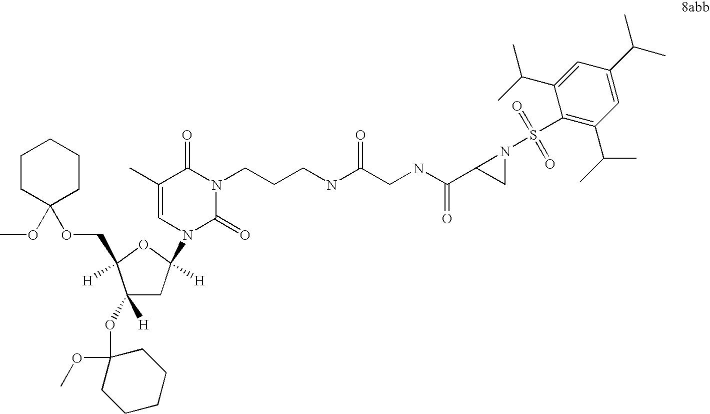

60 mg (0.15 mmol) 7ab were solved in 4 ml dichloromethane followed by 46 μl (0.29 mmol) DIC and 76.5 mg (0.15 mmol) 3-(3-Amino-propyl)-1-[(2R,4S,5R)-4-(1-methoxy-cyclohexyloxy)-5-(1-methoxy-cyclohexyloxymethyl)-tetrahydro-furan-2-yl]-5-methyl-1H-pyrimidine-2,4-dione. The reaction mixture was stirred over night at room temperature and concentrated. The residue was purified by chromatography on silica gel to give 87 mg (65%) of 8abb.

¹H-NMR (CDCl₃): δ=7.56 (s, 1H), 7.21 (s, 2H), 7.01 (t, 1H), 6.72 (t, 1H), 6.35 (t, 1H), 4.51 (m, 1H), 4.28 (sept, 2H), 4.16 (m, 1H), 4.08 (dd, 1H), 3.97 (m, 2H), 3.76 (dd, 1H), 3.67 (dd, 1H), 3.57 (dd, 1H), 3.44 (dd, 1H), 3.21 (s, 3H), 3.18 (s, 3H), 3.16 (m, 2H), 2.92 (sept, 1H), 2.86 (d, 1H), 2.66 (d, 1H), 2.36 (m, 1H), 2.05 (dd, 1H), 1.91 (s, 3H), 1.82-1.76 (m, 6H), 1.54-1.37 (m, 14H), 1.30-1.26 (div. d, 18H) ppm.

### Preparation of Model Compounds to Test Fluorination

### Preparation of 1-trityl-aziridine-2-carboxylic acid benzylamide 9a

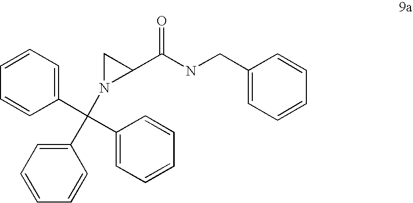

6 g (14.07 mmol) 4a was solved in 300 ml dichloromethane, followed by the addition of 1.57 ml (14.07 mmol) benzylamine. The reaction mixture was stirred overnight at room temperature and concentrated. The residue was purified by chromatography on silica gel to give 3.27 (55%) of 9a.

¹H-NMR (CDCl₃): δ=7.43-7.20 (m, 20H), 7.12 (t, 1H), 4.76 (dd, 1H), 4.35 (dd, 1H), 2.09 (dd, 1H), 2.02 (d, 1H), 1.52 (d, 1H) ppm.

### Preparation of 1-(Toluene-4-sulfonyl)aziridine-2-carboxylic acid benzylamide 10aa

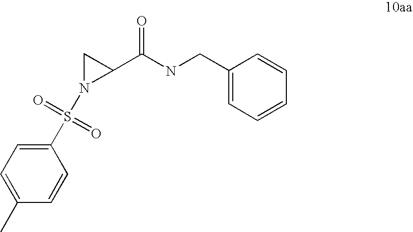

220 mg (0.53 mmol) 9a was solved in chloroform, cooled down to 0° C. and titrated with trifluoro acetic acid until complete conversion. Saturated sodium bicarbonate solution was added until pH 6-7 was reached and the solution was concentrated. The residue was taken up in 15 ml ethyl acetate, treated with 15 ml saturated sodium bicarbonate solution followed by (1.05 mmol) sulfonic acid chloride. The reaction mixture was stirred overnight at room temperature. The organic phase was separated, dried over sodium sulphate and concentrated. The residue was purified by chromatography on silica gel to give (43-65%) of 10aa.

¹H-NMR (CDCl₃): δ=7.81 (d, 2H), 7.36 (d, 2H), 7.29-7.26 (m, 4H), 7.10 (dd, 2H), 6.41 (bt, 1H), 4.36 (dd, 1H), 3.30 (dd, 1H), 2.93 (d, 1H), 2.47 (s, 3H), 2.41 (d, 1H) ppm.

### Preparation of 1-(2,4,6-Tri isopropyl-benzenesulfonyl)-aziridine-2-carboxylic acid benzyl amide 10ab

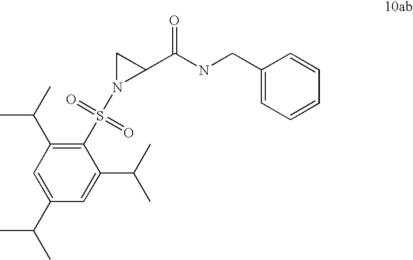

This compound was prepared in an analogous way to 10aa.

¹H-NMR (CDCl₃): δ=7.35-7.27 (m, 4H), 7.17 (s, 2H), 7.15 (m, 1H), 6.32 (t, 1H), 4.37 (dd, 1H), 4.35 (dd, 1H), 4.20 (sept, 2H), 3.42 (dd, 1H), 2.91 (sept, 1H), 2.87 (d, 1H), 2.38 (d, 1H), 1.26 (d, 6H), 1.19 (2d, 12H) ppm.

### Preparation of 1-(3,4-dimethoxy-benzenesulfonyl)-aziridine-2-carboxylic acid benzyl amide 10ac

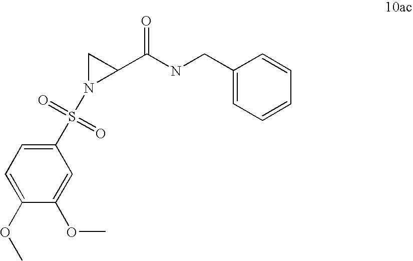

This compound was prepared in an analogous way to 10aa.

¹H-NMR (CDCl₃): δ=7.51 (dd, 1H), 7.34-7.26 (m, 5H), 7.11-7.08 (m, 1H), 6.97 (d, 1H), 6.41 (bt, 1H), 4.39 (dd, 1H), 4.33 (dd, 1H), 3.97 (s, 3H), 3.89 (s, 3H), 3.29 (dd, 1H), 2.83 (d, 1H), 2.42 (d, 1H) ppm.

### Fluorination of Model Compounds

### Preparation of N-Benzyl-3-fluoro-2-(toluene-4-sulfonylamino)-propionamide 11aa

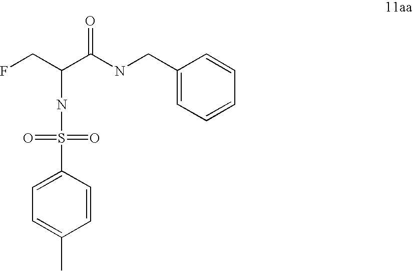

0.079 mmol of 10aa was solved in DMSO followed by the addition of 32.75 mg (0.087 mmol) Kryptofix 222 and 5.05 mg (0.87 mmol) KF. The reaction mixture was stirred at 50°-80° C. for 1 h, taken up in ethyl acetate and extracted with saturated ammonium chloride solution. The combined aqueous phases were extracted twice with ethyl acetate. The combined organic phases were washed with brine, dried over sodium sulphate, filtered and concentrated. The residue was purified by chromatography on silica gel to give (23-71%) of 11aa.

¹H-NMR (CDCl₃): δ=7.76 (d, 2H), 7.38-7.26 (m, 5H), 7.19 (d, 2H), 6.72 (bt, 1H), 5.41 (d, 1H), 4.84 (ddd, 1H), 4.45 (dd, 1H), 4.40 (dd, 1H), 4.20 (ddd, 1H), 3.95 (m, 1H), 2.44 (s, 3H) ppm.

### Preparation of N-Benzyl-3-fluoro-2-(2,4,6-triisopropyl-benzenesulfonylamino)-propionamide 11ab

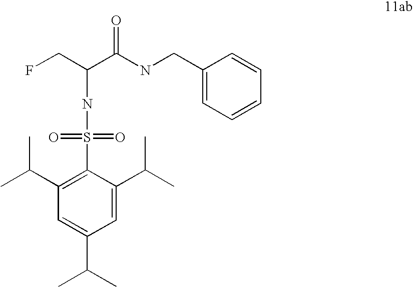

This compound was prepared in an analogous way to 11aa.

¹H-NMR (CDCl₃): δ=7.35-7.16 (m, 7H), 6.84 (bt, 1H), 5.40 (d, 1H), 4.90 (ddd, 1H), 4.51 (dd, 1H), 4.40 (dd, 1H), 4.25 (ddd, 1H), 4.06 (m, 1H), 4.02 (sept, 2H), 2.91 (sept, 1H), 1.19 (m, 18H) ppm.

### Preparation of N-Benzyl-2-(3,4-dimethoxy-benzenesulfonylamino)-3-fluoro-propionamide 11ac

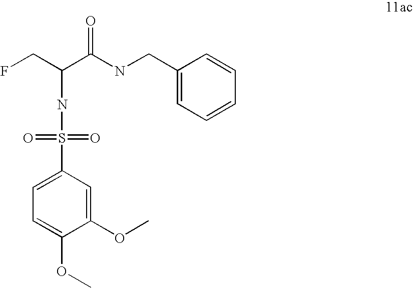

This compound was prepared in an analogous way to 11aa.

¹H-NMR (CDCl₃): δ=7.48 (dd, 1H), 7.34-7.26 (m, 5H), 7.18 (d, 1H), 6.92 (d, 1H), 6.71 (bt, 1H), 5.43 (d, 1H), 4.84 (ddd, 1H), 4.45 (dd, 1H), 4.41 (dd, 1H), 4.26 (ddd, 1H), 3.95 (s, 3H), 3.93 (m, 1H), 3.90 (s, 3H) ppm.

### Fluorination

### Preparation of 3-Fluoro-2-(2,4,6-triisopropyl-benzenesulfonylamino)-propion-Gly-Val-βAla-Phe-Gly-amide 32aba

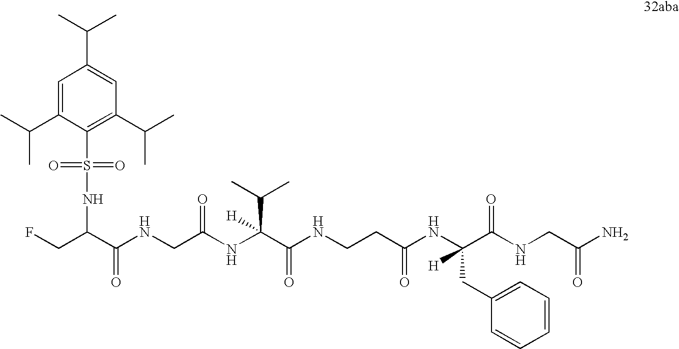

4 mg (5.1 μM) aziridine 8aba were treated with a mixture of 1.2 mg (20.4 μM) KF and 7.7 mg (20.4 μM) Kryptofix in 0.5 ml DMSO for 15 min at 50° C. Then the reaction mixture was analyzed by HPLC-MS which showed a conversion of 10% to the desired product 32aba.

HPLC-MS (ES+): m/z (%)=804.14 (100).

### Preparation of 3-Fluoro-N-[(3-{3-[(2R,4S,5R)-4-(1-methoxy-cyclohexyloxy)-5-(1-methoxy-cyclohexyloxymethyl)-tetrahydro-furan-2-yl]-5-methyl-2,6-dioxo-3,6-dihydro-2H-pyrimidin-1-yl}-propylcarbamoyl)-methyl]-2-(toluene-4-sulfonylamino)-propionamide 32abb

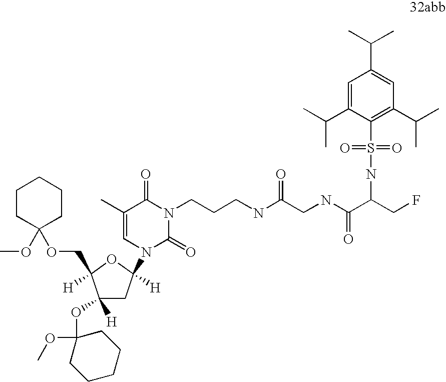

30 mg (0.033 mmol) 8abb were solved in 1.5 ml DMSO followed by 13.6 mg (0.036 mmol) Kryptofix K222 and 2.1 mg (0.036 mml) KF. The reaction mixture was stirred at 50° C. for 30 min until complete conversion of the starting material. The mixture was then diluted with ethyl acetate, washed with saturated aqueous ammonium chloride solution, brine, dried over sodium sulphate, filtrated and concentrated. The residue was purified by chromatography on silica gel to give 5 mg (16%) of 32abb.

¹H-NMR (CDCl₃): δ=7.62 (s, 1H), 7.52 (dd, 1H), 7.30 (t, 1H), 7.18 (s, 2H), 6.84 (d, 1H), 6.34 (dd, 1H), 4.89 (ddd, 1H), 4.51 (m, 1H), 4.42 (ddd, 1H), 4.32 (dd, 1H), 4.19-4.15 (m, 2H), 4.11 (sept, 2H), 4.01-3.97 (m, 2H), 3.71-3.66 (m, 2H), 3.57 (dd, 1H), 3.30 (m, 1H), 3.21 (s, 3H), 3.18 (s, 3H), 3.02 (m, 1H), 2.91 (sept, 1H), 2.40 (ddd, 1H), 2.07-2.03 (m, 2H), 1.88 (s, 3H), 1.86-1.83 (m, 2H), 1.80-1.72 (m, 4H), 1.60-1.45 (m, 16H), 1.27-1.24 (div. d, 18H) ppm.

### Example 2

### Preparation of Compounds Having General Chemical Formula II and Corresponding Model Compounds

Preparation According to Scheme 2 with n=1.

### Preparation of (2-Chlorosulfonyl-3,5-dimethoxy-phenyl)-acetic acid methyl ester 14a

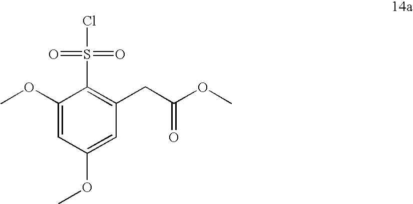

0.4 ml (6 mmol) Chlorosulfonic acid were solved in 4 ml dichloromethane at −10° C. followed by the slow addition of 600 mg (2.85 mmol) (3,5-Dimethoxy-2-methyl-phenyl)-acetic acid methyl ester 13a solved in 2 ml dichloromethane. The reaction mixture was stirred for 1 h at room temperature, diluted with 50 ml acetic acid ethyl ester and washed with 10 ml saturated sodium bicarbonate solution. The phases were separated and the aqueous phase was extracted with acetic acid ethyl ester. The combined organic phases were washed with brine, dried over sodium sulphate, filtrated and concentrated to give 451 mg (51%) of crude 14a which was used in the next step without further purification.

¹H-NMR (CDCl₃): δ=6.54 (d, 1H), 6.37 (d, 1H), 4.03 (s, 5H), 3.89 (s, 3H), 3.72 (s, 3H).

### Preparation of [2-(Aziridine-1-sulfonyl)-3,5-dimethoxy-phenyl]-acetic acid methyl ester 15a

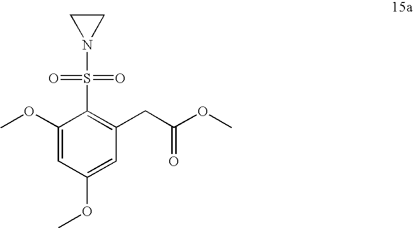

0.22 ml (4.2 mmol) aziridine were solved at 0° C. in a mixture of 3.5 ml saturated sodium bicarbonate solution and 7 ml ethyl acetate followed by the addition of 432 mg (1.4 mmol) (2-Chlorosulfonyl-3,5-dimethoxy-phenyl)-acetic acid methyl ester 14a. The reaction mixture was then stirred at room temperature for 1 h. The phases were separated and the aqueous phase was extracted with ethyl acetate. The combined organic phases were washed with brine, dried over sodium sulphate, filtrated and concentrated. The residue was purified by chromatography on silica gel to give 307 mg (70%) of 15a.

¹H-NMR (CDCl₃): δ=6.52 (d, 1H), 6.38 (d, 1H), 4.05 (s, 2H), 3.95 (s, 3H), 3.86 (s, 3H), 3.71 (s, 3H), 2.44 (s, 4H) ppm.

### Example 3

### Preparation of Compounds Having General Chemical Formula III and Corresponding Model Compounds

Preparation According to Scheme 3 with n=1.

### Preparation of 4-(2,5-Dihydro-pyrrol-1-yl)-4-oxo-butyric acid methyl ester 22a

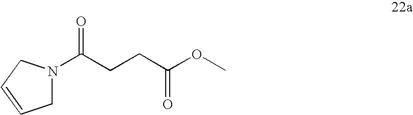

1 g (22.14 mmol) 2,5-dihydro pyrrole 20 was solved in 60 ml dichloromethane and cooled down to 0° followed by the slow addition of 3.3 ml (26.57 mmol) methyl 4-chloro-4-oxobutyrate 21a and 4.6 ml (33.21 mmol) triethylamine. The reaction mixture was stirred at room temperature for 2 h and concentrated. The residue was purified by chromatography on silica gel to give 2.42 g (60%) of 22a.

¹H-NMR (CDCl₃): δ=5.86 (m, 1H), 5.80 (m, 1H), 4.28-4.21 (m, 4H), 3.69 (s, 3H), 2.70 (m, 2H), 2.57 (m, 2H) ppm.

### Preparation of 4-(6-Oxa-3-aza-bicyclo[3.1.0]hex-3-yl)-4-oxo-butyric acid methyl ester 23a

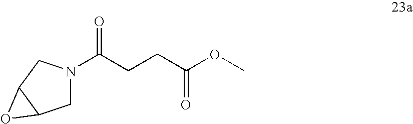

2.24 g (12.23 mmol) 22a was solved in 70 ml dichloromethane followed by the addition of 4.9 g (22.01 mmol, 77%) mCPBA. The reaction mixture was stirred at room temperature for 4 d, diluted with ethyl acetate, washed with bicarbonate and brine, dried over sodium sulphate, filtered and concentrated. The residue was purified by chromatography on silica to give 1.34 g (55%) of 23a.

¹H-NMR (CDCl₃): δ=4.01 (d, 1H), 3.86 (d, 1H), 3.82 (dd, 1H), 3.78 (dd, 1H), 3.73 (s, 3H), 3.62 (d, 1H), 3.44 (d, 1H), 2.82-2.52 (m, 4H) ppm.

### Preparation of 4-((3S,4S)-3-Azido-4-hydroxy-pyrrolidin-1-yl)-4-oxo-butyric acid methyl ester 24a

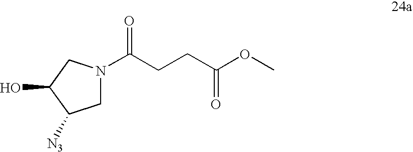

7.6 g (38.15 mmol) epoxide 23a was solved in 250 ml DMF and treated with 3.47 g (53.41 mmol) sodium azide. The reaction mixture was stirred at 100° C. for 5 h, cooled down, diluted with dichloromethane, washed with water and brine, dried over sodium sulphate, filtered and concentrated to give 4.6 g (50%) of crude 24a which was used without further purification.

¹H-NMR (CDCl₃, mixture of diastereomers): δ=4.26 (m, 1H), 4.03 (m, 1H), 3.88-3.44 (m, 4H), 3.67 (s, 3H), 2.70-2.51 (m, 4H) ppm.

### Preparation of 4-[(3S,4S)-3-Azido-4-(toluene-4-sulfonyloxy)-pyrrolidin-1-yl]-4-oxo-butyric acid methyl ester 25a

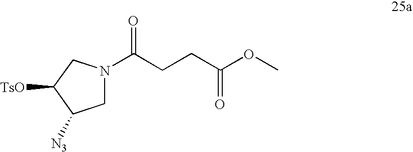

3.91 g (16.14 mmol) 24a was solved in dichloromethane, cooled down to 0° C. followed by the addition of 5.6 ml (40.35 mmol) triethylamine, 590 mg (4.84 mmol) DMAP and 5.39 g (28.25 mmol) tosyl chloride. The reaction mixture was stirred at room temperature overnight, concentrated, taken up in ethyl acetate, washed with saturated ammonium chloride solution and brine, dried over sodium sulphate, filtered and concentrated. The residue was purified by chromatography on silica gel to give 4.07 g (64%) of 25a.

¹H-NMR (CDCl₃, mixture of diastereomers): δ=7.82 (d, 2H), 7.79 (d, 2H), 7.41 (d, 2H), 7.38 (d, 2H), 4.83 (m, 1H), 4.76 (m, 1H), 4.33 (m, 1H), 4.13 (m, 1H), 3.83-3.79 (m, 2H), 3.68 (s, 6H), 3.70-3.53 (m, 6H), 2.66 (m, 4H), 2.56-2.46 (m, 4H), 2.48 (s, 3H), 2.47 (s, 3H) ppm.

### Preparation of 4-(3,6-Diaza-bicyclo[3.1.0]hex-3-yl)-4-oxo-butyric acid methyl ester 26a

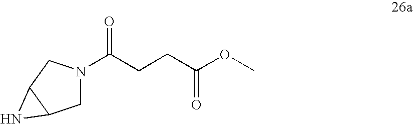

820 mg (2.07 mol) 25a were solved in 32 ml acetonitrile followed by 564 mg (2.14 mmol) triphenyl phosphine. The reaction mixture was stirred at room temperature for 2.5 h followed by the addition of 0.9 ml (49 mmol) water. After stirring at room temperature overnight 0.8 ml (5.77 mmol) triethyl amine were added and the mixture was stirred another 5 h at room temperature and then concentrated. The residue was purified by chromatography on NH2-silica gel to give 263 mg (64%) of 26a.

¹H-NMR (CDCl₃): δ=3.91 (d, 1H), 3.74 (d, 1H), 3.68 (s, 3H), 3.55 (d, 1H), 3.42 (d, 1H), 2.80-2.72 (bm, 2H), 2.65 (dd, 2H), 2.51 (dd, 2H) ppm.

### Preparation of 4-Oxo-4-[6-(2,4,6-triisopropyl-benzenesulfonyl)-3,6-diaza-bicyclo[3.1.0]hex-3-yl]-butyric acid methyl ester 27a

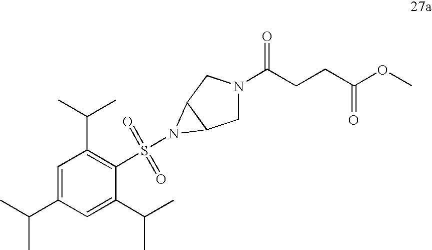

250 mg (1.26 mmol) 26a was solved in 24 ml ethyl acetate and 24 ml saturated sodium bicarbonate solution followed by 764 mg (2.52 mmol) 2,4,6-triisopropyl phenyl sulfonyl chloride. The reaction mixture was stirred over night followed by phase separation and extraction of the aqueous phase with ethyl acetate. The combined organic phases were washed with brine, dried over sodium sulphate, filtrated and concentrated. The residue was purified by chromatography on silica to give 265 mg (45%) of 27a.

¹H-NMR (CDCl₃): δ=7.18 (s, 2H), 4.28 (sept, 2H), 3.94 (d, 1H), 3.79 (d, 1H), 3.70 (dd, 1H), 3.67 (s, 3H), 3.62 (dd, 1H), 3.58 (dd, 1H), 3.42 (dd, 1H), 2.91 (sept, 1H), 2.72-2.40 (m, 4H), 1.27-1.24 (m, 18H) ppm.

### Preparation of 4-Oxo-4-[6-(2,4,6-triisopropyl-benzenesulfonyl)-3,6-diaza-bicyclo[3.1.0]hex-3-yl]-butyric acid 28a

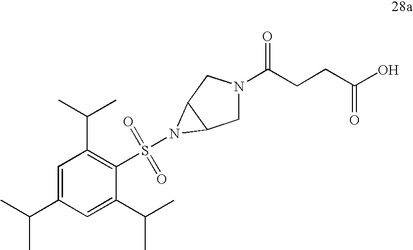

30 mg (0.065 mmol) 27a were solved in 1 ml THF, cooled down to 0° C. and treated with 0.045 ml 2N NaOH. The reaction mixture was stirred at room temperature for 5 h, concentrated, diluted with water and the pH was adjusted at 4 with 10% aqueous citric acid. The aqueous solution was extracted several times with ethyl acetate. The combined organic phases were washed with brine, dried over sodium sulphate and concentrated to give 28 mg (96%) of 28a which was used without further purification.

¹H-NMR (CDCl₃): δ=7.18 (s, 2H), 4.27 (sept, 2H), 3.95 (d, 1H), 3.79 (d, 1H), 3.70 (dd, 1H), 3.62 (dd, 1H), 3.58 (dd, 1H), 3.45 (dd, 1H), 2.91 (sept, 1H), 2.70-2.45 (m, 4H), 1.27-1.24 (m, 18H) ppm.

### Preparation of 4-Oxo-4-[6-(2,4,6-triisopropyl-benzenesulfonyl)-3,6-diaza-bicyclo[3.1.0]hex-3-yl]-butyric acid 2,5-dioxo-pyrrolidin-1-yl ester 29a

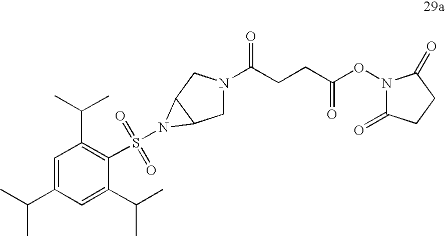

180 mg (0.4 mmol) 28a were solved in 3.6 ml dichloromethane followed by the addition of 265 mg (0.6 mmol) BOP and 50.6 mg (0.44 mmol) N-Hydroxysuccinimide. The reaction mixture was cooled down to 0° C. followed by the addition of 0.12 ml (0.72 mmol) diisopropyl ethyl amine. The mixture was stirred at room temperature over night, diluted with dichloromethane, washed with 10% citric acid, saturated aqueous bicarbonate solution and brine, dried over sodium sulphate, filtrated and concentrated. The residue was purified by chromatography on silica gel to give 85 mg (39%) of 29a.

¹H-NMR (CDCl₃): δ=7.17 (s, 2H), 4.27 (sept, 2H), 3.97 (d, 1H), 3.77 (d, 1H), 3.70 (dd, 1H), 3.62-3.58 (m, 2H), 3.42 (dd, 1H), 2.99-2.87 (m, 3H), 2.83 (s, 4H), 2.58 (m, 2H), 1.28-1.22 (div. d, 18H) ppm.

### Preparation of N-Benzyl-4-oxo-4-[6-(2,4,6-tri isopropyl-benzenesulfonyl)-3,6-diaza-bicyclo[3.1.0]hex-3-yl]-butyramide 30aa

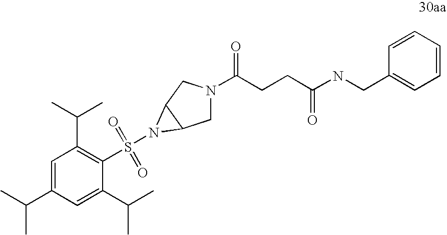

A: 96 mg (0.18 mmol) 29a were solved in 2 ml DMF followed by the addition of 0.019 ml (0.18 mmol) benzyl amine. The reaction mixture was stirred over night at room temperature and concentrated. The residue was purified by chromatography on silica gel to give 31 mg (30%) of 30aa.

B: 78 mg (0.17 mmol) 28a were solved in 4 ml dichloromethane followed by the addition of 84.2 mg (0.19 mmol) BOP and 18.9 μl (0.17 mmol) benzyl amine. The mixture was cooled down to 0° C. and 0.044 ml (0.26 mmol) diisopropyl ethyl amine was added. The reaction mixture was stirred over night at room temperature, diluted with dichloromethane, washed with washed with 10% citric acid, saturated aqueous bicarbonate solution and brine, dried over sodium sulphate, filtrated and concentrated. The residue was purified by chromatography on silica gel to give 68 mg (73%) of 30aa.

¹H-NMR (CDCl₃): δ=7.33-7.23 (m, 5H), 7.17 (s, 2H), 6.31 (bt, 1H), 4.39 (d, 2H), 4.27 (sept, 2H), 3.90 (d, 1H), 3.77 (d, 1H), 3.67 (dd, 1H), 3.63-3.57 (m, 2H), 3.36 (dd, 1H), 2.91 (sept, 1H), 2.61-2.43 (m, 4H), 1.28-1.21 (div. d, 18H) ppm.

### Preparation of 4-Oxo-4-[6-(2,4,6-triisopropyl-benzenesulfonyl)-3,6-diaza-bicyclo[3.1.0]hex-3-yl]-butyric acid-βAla-Phe-amide 30ab

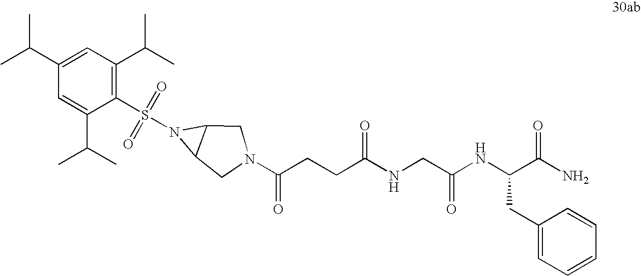

30 mg (0.067 mmol) 28a were solved in 1 ml dichloromethane and 0.2 ml DMF followed by the addition of 10.42 μl (0.067 mmol) DIC and 18.7 mg (0.067 mmol) dipeptide. The reaction mixture was stirred over night at room temperature and concentrated. The residue was purified by chromatography on silica gel to give 21 mg (48%) of 30ab.

MS (ES+): m/z (%)=654 (100).

### Preparation of 4-Oxo-4-[6-(2,4,6-triisopropyl-benzenesulfonyl)-3,6-diaza-bicyclo[3.1.0]hex-3-yl]-butyric acid 3-(3-Amino-propyl)-1-[(2R,4S,5R)-4-(1-methoxy-cyclohexyloxy)-5-(1-methoxy-cyclohexyloxymethyl)-tetrahydro-furan-2-yl]-5-methyl-1H-pyrimidine-2,4-dione 30ac

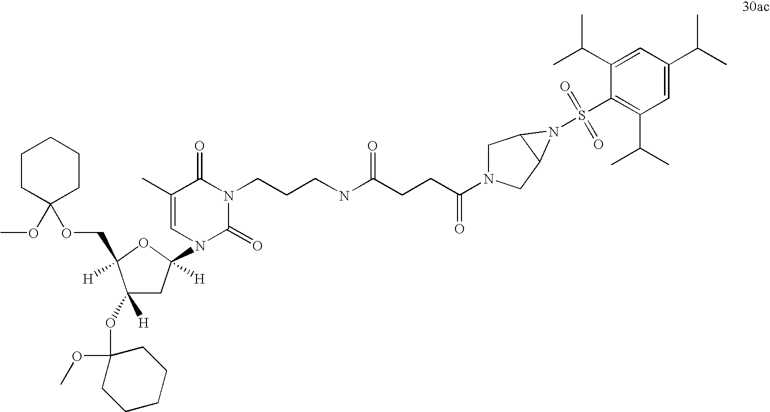

50 mg (0.11 mmol) 28a were solved in 1.5 ml dichloromethane followed by the addition of 26.1 μl (0.17 mmol) DIC. After 30 min 58.1 mg (0.11 mmol) 3-(3-Amino-propyl)-1-[(2R,4S,5R)-4-(1-methoxy-cyclohexyloxy)-5-(1-methoxy-cyclohexyloxymethyl)-tetrahydro-furan-2-yl]-5-methyl-1H-pyrimidine-2,4-dione solved in 1 ml dichloromethane were added. The reaction mixture was stirred at room temperature over night, concentrated and the residue was purified by chromatography on silica gel to give 61 mg (57%) of 30ac.

¹H-NMR (MeOD): δ=7.68 (s, 1H), 7.30 (s, 2H), 6.32 (t, 1H), 4.60 (m, 1H), 4.37 (sept, 2H), 4.19 (dd, 1H), 3.98 (t, 2H), 3.89 (d, 1H), 3.85-3.68 (m, 5H), 3.64 (dd, 2H), 3.43 (d, 1H), 3.24 (s, 6H), 3.20 (t, 2H), 2.97 (sept, 1H), 2.59-2.23 (m, 6H), 1.95 (s, 3H), 1.84-1.44 (m, 23H), 1.30-1.24 (div. d, 18H) ppm.

### Fluorination

### Preparation of N-Benzyl-4-[3-fluoro-4-(2,4,6-tri isopropyl-benzenesulfonylamino)-pyrrolidin-1-yl]-4-oxo-butyramide 35aa

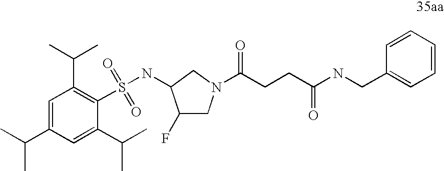

12 mg (0.022 mmol) 30aa were solved in 0.7 ml DMSO followed by the addition of 1.42 mg (0.024 mmol) KF and 9.21 mg (0.024 mmol) Kryptofix K222. The reaction mixture was stirred at 50° C. for 1 h, diluted with saturated aqueous ammonium chloride solution and extracted with ethyl acetate. The combined organic phases were washed with brine, dried over sodium sulphate, filtrated and concentrated. The residue was purified by chromatography on silica gel to give 6 mg (48%) of 35aa.

MS (ESI+): m/z (%)=560 (100), 257 (18).

### Preparation of N-[((S)-1-Carbamoyl-2-phenyl-ethylcarbamoyl)-methyl]-4-[(3S,4S)-3-fluoro-4-(2,4,6-triisopropyl-benzene-sulfonylamino)-pyrrolidin-1-yl]-4-oxo-butyramide 35ab

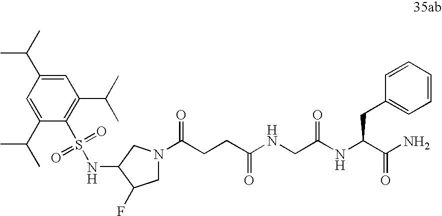

17 mg (0.026 mmol) 30ab were solved in 0.8 ml DMSO followed by the addition of 1.66 mg (0.029 mmol) KF and 10.77 mg (0.029 mmol) Kryptofix K222. The reaction mixture was stirred at 50° C. for 3 h, diluted with saturated aqueous ammonium chloride solution and extracted with ethyl acetate. The combined organic phases were washed with brine, dried over sodium sulphate, filtrated and concentrated. The residue was purified by chromatography on silica gel to give 7.8 mg (44.5%) of 35ab.

MS (ESI+): m/z (%)=674 (100), 658 (57).

### Preparation of 3-(3-Amino-propyl)-1-[(2R,4S,5R)-4-(1-methoxy-cyclohexyloxy)-5-(1-methoxy-cyclohexyloxymethyl)-tetrahydro-furan-2-yl]-5-methyl-1H-pyrimidine-2,4-dione 4-[3-fluoro-4-(2,4,6-triisopropyl-benzenesulfonylamino)-pyrrolidin-1-yl]-4-oxo-butyramide 35ac

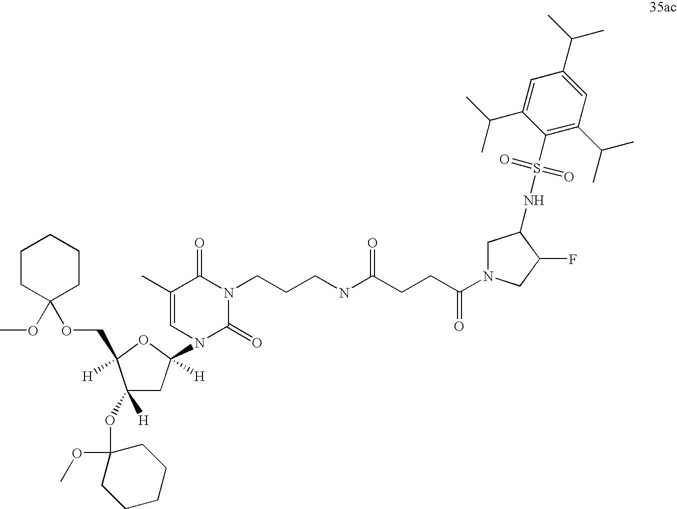

20 mg (0.021 mmol) 30ac were solved in 0.7 ml DMSO followed by the addition of 1.34 mg (0.023 mmol) KF and 8.66 mg (0.023 mmol) Kryptofix K222. The reaction mixture was stirred at 50° C. for 3 h, diluted with saturated aqueous ammonium chloride solution and extracted with ethyl acetate. The combined organic phases were washed with brine, dried over sodium sulphate, filtrated and concentrated. The residue was purified by chromatography on silica gel to give 5.6 mg (27%) of 35ac.

MS (ESI+): m/z (%)=977 (10), 832 (47), 135 (100).

### Radiochemistry

### General Radiolabelling Method

### 1. Model Compounds and Thymidine Derivatives

¹⁸F-Fluoride was azeotropically dried in the presence of Kryptofix 222 (5 mg in 1 ml MeCN) and potassium carbonate (1 mg in 0.5 ml water) or cesium carbonate (2.5 mg in 0.5 ml water) by heating under nitrogen at 100-120° C. for 20-30 minutes. During this time 2-3×1 ml MeCN were added and evaporated under vacuum with a stream of nitrogen to give the dried Kryptofix 222/K₂CO₃ complex or Kryptofix 222/Cs₂CO₃ complex (up to 9.9 GBq). After drying, a solution of the precursor (150-200 μl of 6.8-30 mM in DMSO) was added. The reaction vessel was sealed and heated in the range of 50-90° C. for 15-30 mins to effect labelling. The crude reaction mixture was analyzed by analytical HPLC. The product peak was then confirmed by co-injection of the reaction mixture with the [F-19] cold standard.

### 2. Peptide Containing Natural Histidine

¹⁸F-fluoride was azeotropically dried in the presence of Kryptofix 222 (5 mg in 1 ml MeCN) and cesium carbonate (2.5 mg in 0.5 ml water) by heating under nitrogen at 70-90° C. for 15-30 minutes. During this time 2-3×1 ml MeCN were added and evaporated under vacuum with a stream of nitrogen. After drying, a solution of the precursor (150-200 μl of 7-9 mM in DMSO) was added. The reaction vessel was sealed and heated at 50-90° C. for 15 mins to effect labelling. The crude reaction mixture was analyzed by analytical HPLC. The product peak was then confirmed by co-injection of the reaction mixture with the [F-19] cold standard.

### Additional Points:

i) The solvents could be DMF, DMSO, MeCN, DMA, DMAA, etc., preferably DMSO. The solvents could also be a mixture of solvents as indicated above.
ii) The temperature range could RT-160° C., but preferably in the range 50-90° C.

### Example A

### Radiosynthesis of 3-[¹⁸F]Fluoro-N-benzyl-2-(4-methylphenylsulphonamido)-propanamide 11aa-18F

[¹⁸F]Fluoride was eluted from the QMA Light cartridge (Waters) into a Reactivial (10 ml) with a solution of Kryptofix 222 (5 mg), potassium carbonate (1 mg) in water (500 μl) and MeCN (1 ml). The solvent was removed by heating at 110° C. under vacuum for 10 min with a stream of nitrogen. Anhydrous MeCN (1 ml) was added and evaporated as before. This step was repeated again to give the dried Kryptofix 222/K₂CO₃ complex (2.34 GBq). A solution of N-benzyl-1-tosylaziridine-2-carboxamide 10aa (2 mg) in anhydrous DMSO (200 μl) was added. After heating at 70° C. for 15 min, the reaction was cooled to room temperature and diluted with MeCN (1 ml). The crude reaction mixture was analyzed using an analytical HPLC (Column Nucleosil C18, 250×4 mm, 5 ∥Å, 1 ml/min, solvent A: H₂O, solvent B: MeCN, gradient 10-40% B in 15 mins), the incorporation yield was 95%. The F-18 labelled product was confirmed by co-injection with the F-19 cold standard on the same column.

### Example B

### Radiosynthesis of 3-[¹⁸F]Fluoro-N-benzyl-2-(2,4,6-triisopropylphenylsulphon-amido)-propanamide 11ab-18F

[¹⁸F]Fluoride was eluted from the QMA Light cartridge (Waters) into a Reactivial (10 ml) with a solution of Kryptofix 222 (5 mg), potassium carbonate (1 mg) in water (500 μl) and MeCN (1 ml). The solvent was removed by heating at 110° C. under vacuum for 10 min with a stream of nitrogen. Anhydrous MeCN (1 ml) was added and evaporated as before. This step was repeated again to give the dried Kryptofix 222/K₂CO₃ complex (5.9 GBq). A solution of N-benzyl-1-(2,4,6-triisopropylphenylsulphonyl)-aziridine-2-carboxamide 10ab (2 mg) in anhydrous DMSO (200 μl) was added. After heating at 60° C. for 15 min, the reaction was cooled to room temperature and dilute with MeCN (1 ml). The crude reaction mixture was analyzed using an analytical HPLC (Column Nucleosil C18, 250×4 mm, 5 μÅ, 1 ml/min, solvent A: H₂O, solvent B: MeCN, gradient 40-95% B in 20 mins), the incorporation yield was 97%. The F-18 labelled product was confirmed by co-injection with the F-19 cold standard on the same column.

### Example C

### Radiosynthesis of 3-[¹⁸F]Fluoro-N-benzyl-2-(3,4-dimethoxyphenylsulphon-amido)-propanamide 11ac-18F

[¹⁸F]Fluoride was eluted from the QMA Light cartridge (Waters) into a Reactivial (10 ml) with a solution of Kryptofix 222 (5 mg), potassium carbonate (1 mg) in water (500 μl) and MeCN (1 ml). The solvent was removed by heating at 100° C. under vacuum for 10 min with a stream of nitrogen. Anhydrous MeCN (1 ml) was added and evaporated as before. This step was repeated again to give the dried Kryptofix 222/K₂CO₃ complex (9.9 GBq). A solution of N-benzyl-1-(3,4-dimethoxyphenylsulphonyl)-aziridine-2-carboxamide 10ac (2 mg) in anhydrous DMSO (200 μl) was added. After heating at 70° C. for 15 min, the reaction was cooled to room temperature and diluted with MeCN (1 ml). The crude reaction mixture was analyzed using an analytical HPLC (Column Nucleosil C18, 250×4 mm, 5 μÅ, 1 ml/min, solvent A: H₂O, solvent B: MeCN, gradient 10-60% B in 15 mins), the incorporation yield was 97%. The F-18 labelled product was confirmed by co-injection with the F-19 cold standard on the same column.

### Example D

### Radiosynthesis of N-Benzyl-4-[3-[¹⁸F]fluoro-4-(2,4,6-triisopropyl-benzene-sulfonylamino)-pyrrolidin-1-yl]-4-oxo-butyramide 35aa-18F

[¹⁸F]Fluoride (5 GBq) was eluted from the QMA Light cartridge (Waters) into a Reactivial (10 ml) with a solution of Kryptofix 222 (5 mg), potassium carbonate (1 mg) in water (500 μl) and MeCN (1 ml). The solvent was removed by heating at 110° C. under vacuum for 10 mins with a stream of nitrogen. Anhydrous MeCN (1 ml) was added and evaporated as before. This step was repeated again to give the dried Kryptofix 222/K₂CO₃ complex. A 0.0185 M solution of N-Benzyl-4-oxo-4-[6-(2,4,6-triisopropyl-benzenesu lfonyl)-3,6-diaza-bicyclo[3.1.0]hex-3-yl]-butyramide 30aa (2 mg, 3.7 μmol) in anhydrous DMSO (200 μl) was added. After heating at 90° C. for 15 min, the reaction was cooled to room temperature and dilute with MeCN (1 ml). The crude reaction mixture was analyzed using an analytical HPLC (Column Lichrosorb RP18, 250×4 mm, 5 μÅ, 1 ml/min, solvent A: H₂O, solvent B: MeCN, gradient 40-95% B in 30 mins), the incorporation yield was 96%. The F-18 labelled product was confirmed by co-injection with the F-19 cold standard on the same column.

### Example E

### Radiosynthesis of 3-Fluoro-2-(2,4,6-triisopropyl-benzenesulfonylamino)-propion-Gly-Val-βAla-Phe-Gly-amide 32aba-18F

[¹⁸F]Fluoride (1.78 GBq) was eluted from the QMA Light cartridge (Waters) into a Reactivial (10 ml) with a solution of Kryptofix 222 (5 mg), potassium carbonate (1 mg) in water (500 μl) and MeCN (1 ml). The solvent was removed by heating at 110° C. under vacuum for 10 mins with a stream of nitrogen. Anhydrous MeCN (1 ml) was added and evaporated as before. This step was repeated again to give the dried Kryptofix 222/K₂CO₃ complex. A 0.0127 M solution of 1-(2,4,6-Triisopropyl-benzenesulfonyl)-aziridine-2-carboxylic-Gly-Val-βAla-Phe-Gly-amide 8aba (2 mg) in anhydrous DMSO (200 μl) was added. After heating at 60° C. for 15 min, the reaction was cooled to room temperature and diluted with MeCN (1 ml). The crude reaction mixture was analyzed using an analytical HPLC (Column Lichrosorb RP18, 250×4 mm, 5 μÅ, 1 ml/min, solvent A: H₂O, solvent B: MeCN, gradient 15-95% B in 20 mins), the incorporation yield was 49%. The F-18 labelled product was confirmed by co-injection with the F-19 cold standard on the same column.

### Example F

### Radiosynthesis of 3-Fluoro-N-[(3-{3-[(2R,4S,5R)-4-(1-methoxy-cyclohexyloxy)-5-(1-methoxy-cyclohexyloxymethyl)-tetrahydro-furan-2-yl]-5-methyl-2,6-dioxo-3,6-dihydro-2H-pyrimidin-1-yl}-propylcarbamoyl)-methyl]-2-(toluene-4-sulfonylamino)-propionamide 32abb-18F

[¹⁸F]Fluoride (4.9 GBq) was eluted from the QMA Light cartridge (Waters) into a Reactivial (5 ml) with a solution of Kryptofix 222 (5.5 mg), cesium carbonate (2.5 mg) in water (500 μl) and MeCN (1 ml). The solvent was removed by heating at 110° C. under vacuum for 10 mins with a stream of nitrogen. Anhydrous MeCN (1 ml) was added and evaporated as before. This step was repeated again to give the dried Kryptofix 222/Cs₂CO₃ complex. A 0.011 M solution of 1-(2,4,6-Triisopropyl-benzenesulfonyl)-aziridine-2-carboxylic acid [(3-{3-[(2R,4S,5R)-4-(1-methoxy-cyclohexyloxy)-5-(1-methoxy-cyclohexyloxymethyl)-tetrahydro-furan-2-yl]-5-methyl-2,6-dioxo-3,6-dihydro-2H-pyrimidin-1-yl}-propylcarbamoyl)-methyl]-amide 8abb (2 mg) in anhydrous DMSO (200 μl) was added. After heating at 90° C. for 20 min, the reaction was cooled to room temperature and diluted with MeCN (1 ml). The crude reaction mixture was analyzed using an analytical HPLC (Column Lichrosphere 100 RP18e, 5 μm, 1 ml/min, solvent A: H₂O, solvent B: MeCN, gradient 5-95% in 10 mins+iso95% 10 mins), the incorporation yield was 87%.

The F-18 labelled product was purified through Silica cartridge (Macherey-Nagel) and rinsed with another 1 ml of MeCN. Deprotection step was achieved by adding solution of HCl 1 M (0.5 ml) to purified compound and reaction at ambient temperature for 5 mins. Another injection was done using analytical HPLC, followed by co-injection with the F-19 cold standard in order to confirm the final F-18 labelled product fully deprotected: 87% radiochemically pure.

### Example G

### Radiosynthesis of 3-(3-Amino-propyl)-1-[(2R,4S,5R)-4-(1-methoxy-cyclohexyloxy)-5-(1-methoxy-cyclohexyloxymethyl)-tetrahydro-furan-2-yl]-5-methyl-1H-pyrimidine-2,4-dione 4-[3-fluoro-4-(2,4,6-triisopropyl-benzenesulfonylamino)-pyrrolidin-1-yl]-4-oxo-butyramide 35ac-18F

[¹⁸F]Fluoride (6.94 GBq) was eluted from the QMA Light cartridge (Waters) into a Reactivial (5 ml) with a solution of Kryptofix 222 (5 mg), potassium carbonate (1 mg) in water (500 μl) and MeCN (1 ml). The solvent was removed by heating at 110° C. under vacuum for 10 mins with a stream of nitrogen. Anhydrous MeCN (1 ml) was added and evaporated as before. This step was repeated again to give the dried Kryptofix 222/K₂CO₃ complex. A 0.0104 M solution of 4-Oxo-4-[6-(2,4,6-triisopropyl-benzenesulfonyl)-3,6-diaza-bicyclo[3.1.0]hex-3-yl]-butyric acid 3-(3-Amino-propyl)-1-[(2R,4S,5R)-4-(1-methoxy-cyclohexyloxy)-5-(1-methoxy-cyclohexyloxy-methyl)-tetrahydro-furan-2-yl]-5-methyl-1H-pyrimidine-2,4-dione 30ac (2 mg) in anhydrous DMSO (200 μl) was added. After heating at 90° C. for 15 min, the reaction was cooled to room temperature and diluted with MeCN (1 ml). The crude reaction mixture was analyzed using an analytical HPLC (Column Lichrosphere 100 RP18e, 5 μm, 1 ml/min, solvent A: H₂O, solvent B: MeCN, gradient 5-95% in 10 mins+iso95% 10 mins), the incorporation yield was 83%.

The F-18 labelled product was purified through Silica cartridge (Macherey-Nagel) and rinsed with another 1 ml of MeCN. Deprotection step was achieved by adding solution of HCl 1M (0.5 ml) to purified compound and reaction at ambient temperature for 5 mins. Another injection was done using analytical HPLC, followed by co-injection with the F-19 cold standard in order to confirm the final F-18 labelled product fully deprotected: 100% radiochemically pure.

For the HPLC chromatogram of reaction mixture with co-injection of the cold standard refer to FIG. 1.

### Hydrolytic Stability of the β-Fluoro Amines

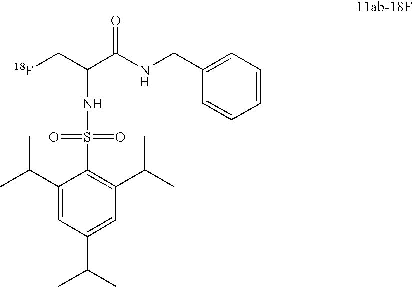

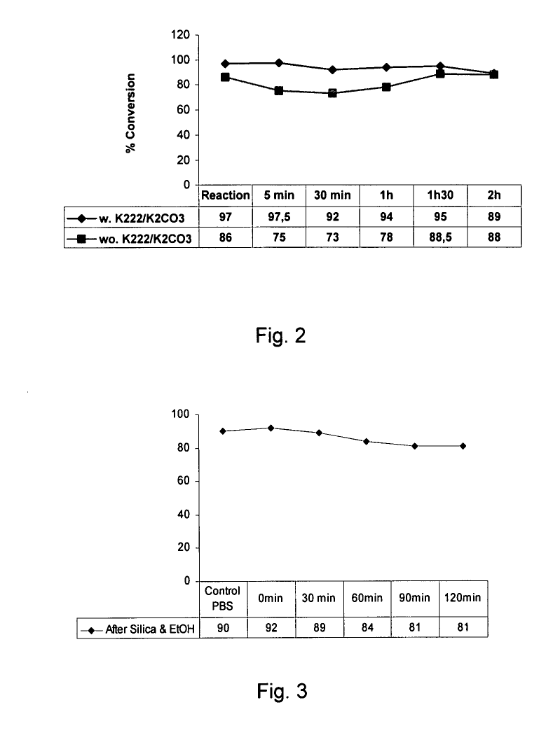

The β-fluoro amino acid derivative 11ab-18F is quite stable under neutral and basic conditions (FIG. 2).

### Plasma Stability of the β-Fluoro Amines

675 μl EtOH were added to the reactive-vial and then 5 aliquots of 70 μl of the Plasma solution were incubated for different time periods.

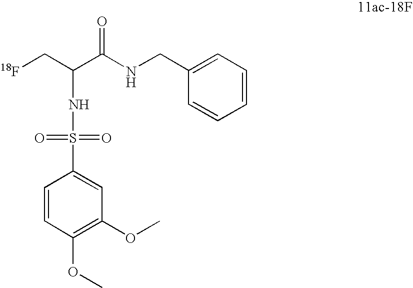

11ac-18F is stable in solution with Human Plasma (FIG. 3).

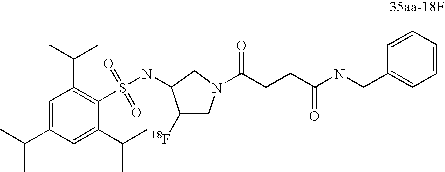

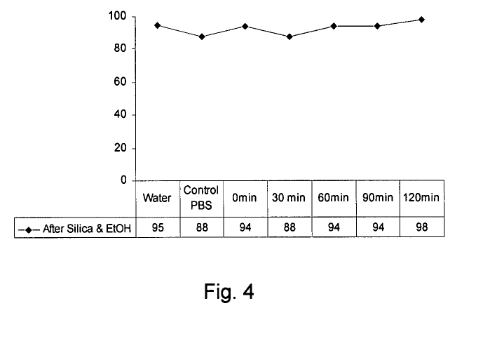

35aa-18F is stable in solution with Human Plasma (FIG. 4).

### In Vitro Binding Affinity

In vitro binding affinity and specificity of Bombesin analogs for the human bombesin 2 receptor (GRPR) were assessed via a competitive receptor-binding assay using ¹²⁵I-[Tyr⁴]-Bombesin (Perkin Elmer; specific activity 81.4 TBq/mmol) as GRPR-specific radioligand. The assay was performed based on the scintillation proximity assay (SPA) technology (J. W. Carpenter et al., Meth. Mol. Biol., 2002; 190:31-49) using GRPR-containing cell membranes (Perkin Elmer) and wheat germ agglutinin (WGA)-coated PVT beads (Amersham Bioscience).

Briefly, GRPR-containing membranes and WGA-PVT beads were mixed in assay buffer (50 mM Tris/HCl pH 7.2, 5 mM MgCl₂, 1 mM EGTA, Complete protease inhibitor (Roche Diagnostics GmbH) and 0.3% PEI) to give final concentrations of approximately 100 μg/ml protein and 40 mg/ml PVT-SPA beads. The ligand ¹²⁵I-[Tyr⁴]-Bombesin was diluted to 0.5 nM in assay buffer. The test compounds were dissolved in DMSO to give 1 mM stock solutions. Later on, they were diluted in assay buffer to 8 pM-1.5 μM.

The assay was then performed as follows: First, 10 μl of compound solution to be tested for binding were placed in white 384 well plates (Optiplate-384, Perkin-Elmer). At next, 20 μl GRPR/WGA-PVT bead mixture and 20 μl of the ligand solution were added. After 90 minutes incubation at room temperature, another 50 μl of assay buffer were added, the plate sealed and centrifuged for 10 min at 520×g at room temperature. Signals were measured in a TopCount (Perkin Elmer) for 1 min integration time per well. The IC₅₀ was calculated by nonlinear regression using the GraFit data analysis software (Erithacus Software Ltd.). Furthermore, the K_{I} was calculated based on the IC₅₀ for test compound as well as the K_{D} and the concentration of the ligand ¹²⁵I-[Tyr⁴]-Bombesin. Experiments were done with quadruple samples.

Synthesis of H—Y-E: Solid-phase peptide synthesis (SPPS) involves the stepwise addition of amino acid residues to a growing peptide chain that is linked to an insoluble support or matrix, such as polystyrene. The C-terminal residue of the peptide is first anchored to a commercially available support (e.g., Rink amide resin) with its amino group protected with an N-protecting agent, fluorenylmethoxycarbonyl (FMOC) group. The amino protecting group is removed with suitable deprotecting agent such as piperidine for FMOC and the next amino acid residue (in N-protected form) is added with a coupling agents such as dicyclohexylcarbodiimide (DCC), di-isopropyl-cyclohexylcarbodiimide (DCCl), hydroxybenzotriazole (HOBt). Upon formation of a peptide bond, the reagents are washed from the support. After addition of the final residue of (Y), the peptide is attached to the solid support is ready for the coupling of RG-L₁-B₁—OH.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever. In the foregoing and in the examples, all temperatures are set forth uncorrected in degrees Celsius and, all parts and percentages are by weight, unless otherwise indicated.

The entire disclosures of all applications, patents and publications, cited herein and of corresponding Europe application Nos. 070900010.4, filed Jan. 9, 2007 and 07090079.0, filed Apr. 23, 2007; and U.S. Provisional Application Nos. 60/880,010 filed Jan. 12, 2007, and 60/914,886, filed Apr. 30, 2007.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound comprising an aziridine ring being appropriately activated for labelling purposes, wherein a targeting agent radical, either directly or via an appropriate linker, is attached either to the aziridine ring or to a five-membered carbocyclic or heterocyclic ring which is fused to the aziridine ring.

2. The compound according to claim 1, having general chemical Formula I 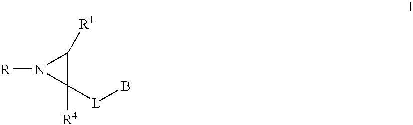
wherein
R represents Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, Ns, Cbz, Bz, Bn, Boc, Fmoc, methoxycarbonyl, ethoxycarbonyl, allyloxycarbonyl, Tr or acyl;
wherein R² represents hydrogen, substituted or non-substituted, linear or branched C₁-C₆ alkyl, cycloalkyl, heterocycloalkyl, aryl, heteroaryl, aralkyl or heteroaralkyl, OH, OR³, NH₂, NHR³, N(R³)₂, SH, SR³, halogen, NO₂, C(═O)R³, C(═O)OR³, C(═O)NHR³ or C(═O)(NR³)₂,
wherein R³ represents hydrogen, substituted or non-substituted, linear or branched C₁-C₆ alkyl, aryl, cycloalkyl, heterocycloalkyl, aryl, heteroaryl, aralkyl or heteroaralkyl;
R¹ and R⁴, independently, are selected from the group comprising hydrogen, substituted and non-substituted, linear and branched C₁-C₆ alkyl, cycloalkyl, heterocycloalkyl, aryl, heteroaryl, aralkyl and heteroaralkyl;
L represents a linker suitable for coupling with the targeting agent radical,
B represents the targeting agent radical,
and a pharmaceutically acceptable salt of an inorganic or organic acid thereof, a hydrate, complex, ester, amide, solvate and prodrug thereof.

3. The compound according to claim 2, wherein
R is Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, or Ns;
wherein R² represents hydrogen, substituted or non-substituted, linear or branched C₁-C₆ alkyl, OH, OR³, NH₂, NHR³, N(R³)₂, SH, SR³, Cl, Br, I, NO₂, C(═O)R³, C(═O)OR³, C(═O)NHR³, C(═O)N(R³)₂; wherein R³ represents hydrogen or substituted or non-substituted, linear or branched C₁-C₆ alkyl, and
R¹ and R⁴, independently, are selected from the group comprising hydrogen and substituted and non-substituted, linear and branched C₁-C₆ alkyl.

4. The compound according to claim 2, wherein
R is 2,4,6-triisopropyl-phenyl-sulfonyl, 3,4-dimethoxy-phenyl-sulfonyl, unsubstituted phenyl-sulfonyl or phenyl-sulfonyl being substituted with 1-5 R² moieties;
wherein R² represents hydrogen, substituted or non-substituted, linear or branched C₁-C₆ alkyl or OR³, wherein R³ represents substituted or non-substituted, linear or branched C₁-C₆ alkyl, and
R¹ and R⁴ represent hydrogen.

5. The compound according to claim 1, having general chemical Formula II 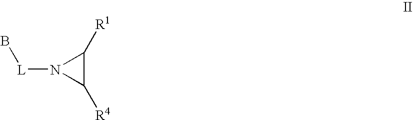
wherein
R¹ and R⁴, independently, are selected from the group comprising hydrogen, substituted and non-substituted, linear and branched C₁-C₆ alkyl, cycloalkyl, heterocycloalkyl, aryl, heteroaryl, aralkyl and heteroaralkyl;
L represents a linker suitable for coupling with the targeting agent radical and for appropriate activation of the aziridine ring;
B represents the targeting agent radical
and a pharmaceutically acceptable salt of an inorganic or organic acid thereof, a hydrate, complex, ester, amide, solvate and prodrug thereof.

6. The compound according to claim 5, wherein
R¹ and R⁴, independently, are selected from the group comprising hydrogen and substituted and non-substituted, linear and branched C₁-C₆ alkyl.

7. The compound according to claim 1, having general chemical Formula III: 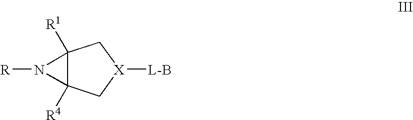
wherein
R represents Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, Ns, Cbz, Bz, Bn, Boc, Fmoc, methoxycarbonyl, ethoxycarbonyl, allyloxycarbonyl, Tr or acyl;
wherein R² represents hydrogen, substituted or non-substituted, linear or branched C₁-C₆ alkyl, cycloalkyl, heterocycloalkyl, aryl, heteroaryl, aralkyl, or heteroaralkyl, OH, OR³, NH₂, NHR³, N(R³)₂, SH, SR³, Cl, Br, I, NO₂, C(═O)R³, C(═O)OR³, C(═O)NHR³ or C(═O)N(R³)₂;
wherein R³ represents hydrogen, substituted or non-substituted, linear or branched C₁-C₆ alkyl, aryl, cycloalkyl, heterocycloalkyl, aryl, heteroaryl, aralkyl, or heteroaralkyl;
R¹ and R⁴, independently, are selected from the group comprising hydrogen, substituted and non-substituted, linear and branched C₁-C₆ alkyl, cycloalkyl, heterocycloalkyl, aryl, heteroaryl, aralkyl and heteroaralkyl;
X represents N or C substituted by hydrogen;
L represents a linker suitable for coupling with the targeting agent radical;
B represents the targeting agent radical,
and a pharmaceutically acceptable salt of an inorganic or organic acid thereof, a hydrate, complex, ester, amide, solvate and prodrug thereof.

8. The compound according to claim 7, wherein
R is Ts, 2,4,6-triisopropyl-phenyl-sulfonyl, 3,4-dimethoxy-phenyl-sulfonyl, unsubstituted phenyl-sulfonyl, phenyl-sulfonyl being substituted with 1-5 R² moieties, or Ns;
wherein R² represents hydrogen, substituted or non-substituted, linear or branched C₁-C₆ alkyl, OH, OR³, NH₂, NHR³, N(R³)₂, SH, SR³, Cl, Br, I, NO₂, C(═O)R³, C(═O)OR³, C(═O)NHR³, C(═O)N(R³)₂; wherein R³ represents hydrogen, substituted or non-substituted, linear or branched C₁-C₆ alkyl or aryl;
R¹ and R⁴, independently, are selected from the group comprising hydrogen and substituted and non-substituted, linear and branched C₁-C₆ alkyl;
X represents N or C substituted by hydrogen.

9. The compound according to claim 7, wherein
R is Ts, 2,4,6-triisopropyl-phenyl-sulfonyl, 3,4-dimethoxy-phenyl-sulfonyl, unsubstituted phenyl-sulfonyl or phenyl-sulfonyl being substituted with 1-5 R² moieties;
wherein R² represents hydrogen, substituted or non-substituted, linear or branched C₁-C₆ alkyl, OR³, SR³, Cl, Br, I, C(═O)R³, C(═O)OR³, C(═O)NHR³ or C(═O)N(R³)₂, wherein R³ represents hydrogen, substituted or non-substituted, linear or branched C₁-C₆ alkyl or aryl;
R¹ and R⁴, independently, are selected from the group comprising hydrogen and substituted and non-substituted, linear and branched C₁-C₆ alkyl; and
X represents N.

10. The compound according to claim 1, comprising
1-(Toluene-4-sulfonyl)-aziridine-2-carboxylic acid-Gly-Val-βAla-Phe-Gly-amide
1-(2,4,6-Triisopropyl-benzenesulfonyl)-aziridine-2-carboxylic-Gly-Val-βAla-Phe-Gly-amide
1-(2,4,6-Triisopropyl-benzenesulfonyl)-aziridine-2-carboxylic acid [(3-{3-[(2R,4S,5R)-4-(1-methoxy-cyclohexyloxy)-5-(1-methoxy-cyclohexyloxymethyl)-tetrahydro-furan-2-yl]-5-methyl-2,6-dioxo-3,6-dihydro-2H-pyrimidin-1-yl}-propylcarbamoyl)-methyl]-amide

11. The compound according to claim 1, wherein the linker -L- is selected from the group comprising substituted and non-substituted, linear and branched C₁-C₆ alkyl, cycloalkyl, alkenyl, heterocycloalkyl, aryl, heteroaryl, substituted aryl, substituted heteroaryl, aralkyl, heteroaralkyl, alkylenoxy, aryloxy, aralkoxy, —C(═O)—, —C(═O)O—, —C(═O)NH—, —C(═O)N—(CH₂)ᵣ—C(═O)— and —C(═O)—(CH₂)ₙ—C(═O)—, —SO₂—, —SO₂NR³—, —NR³SO₂—, —NR³C(═O)O—, —NR³C(═O)NR³—, —NR³—, —NH—NH—, —NH—O—, —(CH₂)ₙ—C(═O)—NR³—CH₂—C(═O)—, —SO₂— (unsubstituted or substituted aryl)-(CH₂)ₙ—C(═O)— 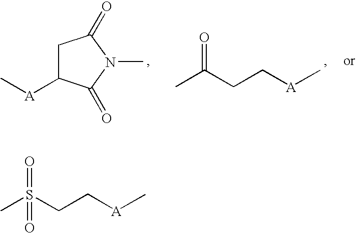
wherein n is from 1 to 3, -A- represents —S— or —NR³—, wherein R³ represents hydrogen, substituted or non-substituted, linear or branched C₁-C₆ alkyl, cycloalkyl, heterocycloalkyl, aryl, heteroaryl, aralkyl or heteroaralkyl.

12. The compound according to claim 1, wherein
-L- is selected from the group comprising linear and branched C₁-C₆ alkylen, -(substituted and unsubstituted, linear and branched C₁-C₆ alkylen)-C(═O)—, —C(═O)—, —C(═O)NH—, —C(═O)N—(CH₂)ₙ—C(═O)— and —C(═O)—(CH₂)ₙ—C(═O) with n=1-3.

13. The compound according to claim 1, wherein the targeting agent radical B comprises biomolecules selected from the group comprising peptides, peptidomimetics, small molecules and oligonucleotides.

14. The compound according to claim 1, wherein the targeting agent B comprises biomolecules selected from the group comprising peptides comprising from 2 to 100 amino acids.

15. A method of preparing a compound according to claim 1 by reacting a suitable precursor molecule with the targeting agent or a precursor thereof.

16. A fluorinated compound obtainable by a ring opening flurination reaction of the aziridine ring of a compound according to claim 1, and a pharmaceutically acceptable salt of an inorganic or organic acid thereof, a hydrate, complex, ester, amide, solvate and prodrug thereof.

17. A fluorinated compound, having any one of general chemical Formulae I-F-A and I-F-B: 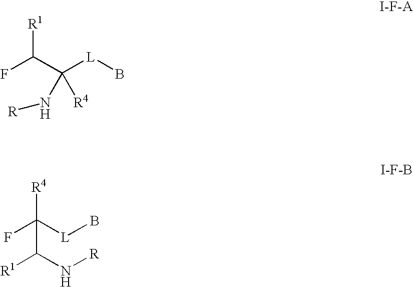
wherein R, R¹, R⁴, L and B have the meanings as given in claim 1 and F is fluorine isotope,
and a pharmaceutically acceptable salt of an inorganic or organic acid thereof, a hydrate, complex, ester, amide, solvate and prodrug thereof.

18. A fluorinated compound, having any one of general chemical Formulae II-F-A and II-F-B: 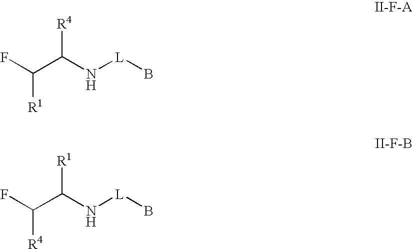
wherein R, R¹, R⁴, L and B have the meanings as given in claim 1 and F is fluorine isotope,
and a pharmaceutically acceptable salt of an inorganic or organic acid thereof, a hydrate, complex, ester, amide, solvate and prodrug thereof.

19. A fluorinated compound, having any one of general chemical Formulae III-F-A and III-F-B: 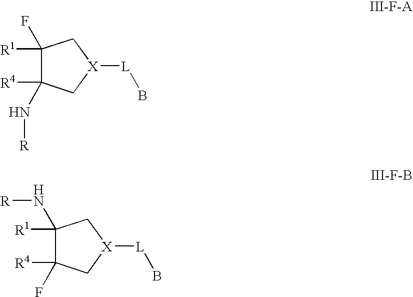
wherein R, R¹, R⁴, L and B have the meanings as given in claim 1 and F is fluorine isotope,
and a pharmaceutically acceptable salt of an inorganic or organic acid thereof, a hydrate, complex, ester, amide, solvate and prodrug thereof.

20. The fluorinated compound according to claim 16, wherein the fluorine isotope is radioactive or non-radioactive isotope, more preferably ¹⁸F or ¹⁹F.

21. The fluorinated compound according to claim 16, wherein the radioactive fluorine isotope is ¹⁸F.

22. A method of preparing a fluorinated compound by reacting a compound according to claim 1 with an appropriate fluorinating agent under appropriate reaction conditions.

23. The method according to claim 22, wherein said fluorinating agent is K¹⁸F, H¹⁸F, KH¹⁸F₂ or a tetraalkyl ammonium salt of ¹⁸F⁻.

24. The method according to claim 22, wherein said fluorinating agent is K¹⁸F.

25. The method according to claim 22, wherein reaction temperature is adjusted to 100° C. or less.

26. The method according to claim 22, wherein reaction temperature is adjusted to 80° C. or less.

27. The method according to claim 22, wherein a solvent is used which is selected from the group comprising DMF, DMSO, MeCN, DMA, DMAA and a mixture thereof.

28. The method according to claim 2, wherein a solvent is used which is DMSO.

29. A composition comprising a compound or a pharmaceutically acceptable salt of an inorganic or organic acid thereof, a hydrate, complex, ester, amide, solvate or prodrug thereof according to claim 1 or a fluorinated compound or a pharmaceutically acceptable salt of an inorganic or organic acid thereof, a hydrate, complex, ester, amide, solvate or prodrug thereof, and a pharmaceutically acceptable carrier, diluent, excipient or adjuvant.

30. A kit comprising a vial containing a predetermined quantity of a compound or a pharmaceutically acceptable salt of an inorganic or organic acid thereof, a hydrate, complex, ester, amide, solvate or prodrug thereof according to claim 1 and a pharmaceutically acceptable carrier, diluent, excipient or adjuvant for the manufacture of a compound.

31. A kit comprising any one of the fluorinated compound or a pharmaceutically acceptable salt of an inorganic or organic acid thereof, a hydrate, complex, ester, amide, solvate or prodrug thereof of claim 16 or a composition comprising the same, e.g., in powder form, and a container containing an appropriate solvent for preparing a solution of said fluorinated compound or composition for administration thereof to an animal, including a human.

32. A use of a fluorinated compound or of a pharmaceutically acceptable salt of an inorganic or organic acid thereof, a hydrate, complex, ester, amide, solvate or prodrug thereof according to claim 16 or of a composition or of a kit thereof for the manufacture of a medicament.

33. A use of a fluorinated compound or of a pharmaceutically acceptable salt of an inorganic or organic acid thereof, a hydrate, complex, ester, amide, solvate or prodrug thereof according to claim 16 a composition or of a kit thereof for the manufacture of a diagnostic imaging agent.

34. The use according to claim 33 wherein the diagnostic imaging agent is for positron emission tomography.

35. The use according to claim 33 for imaging of tumors, imaging of inflammatory and/or neurodegenerative diseases, such as multiple sclerosis of Alzheimer's disease, or imaging of angiogenesis-associates diseases, such as growth of solid tumors, and rheumatoid arthritis.
